(19) United States
(12) Patent Application Publication
(10) Pub. No.: US 2023/0190844 A1
(43) Pub. Date: Jun. 22, 2023

(54) COMPOSITION AND METHOD FOR TREATING CANCER WITH CANNABINOIDS

(71) Applicant: CANNABICS PHARMACEUTICALS INC., Bethesda, MD (US)

(72) Inventors: Eyal BALLAN, Ramat Hasharon (IL); Ilan HOCHMAN, Oranit (IL); Gabriel YARIV, Ramat Hasharon (IL); Eyal BARAD, Kibutz Sdot Yam (IL)

(21) Appl. No.: 17/918,410

(22) PCT Filed: Apr. 13, 2021
(86) PCT No.: PCT/IL2021/050419

§ 371 Date: Oct. 12, 2022

Related U.S. Application Data
(60) Provisional application No. 63/009,071, filed on Apr. 13, 2020.

(51) Int. Cl. A61K 36/185; A61K 45/06; A61P 35/00

(52) U.S. Cl. CPC A61K 36/185; A61K 45/06; A61P 35/00

(57) ABSTRACT

The present invention relates to combinations of cannabinoids selected from cannabichromenic acid (CBCA), cannabidivarin (CBDV), cannabidivarinic acid (CBDVA), tetrahydrocannabivarin (THCV) and cannabigerolic acid (CBGA), and to methods of treating cancer comprising the same.

### TECHNICAL FIELD OF THE INVENTION

The present invention relates to pharmaceutical compositions for treating cancer and methods for treating the same. In particular, the invention relates to compositions and methods of treating colon cancer by using cannabinoids.

### BACKGROUND ART

Cannabinoids bind to human cannabinoid receptors CB-1 and CB-2, thus causing a range of physiological effects in, for example, the nervous system, the immune and bone cells. There are 113 identified cannabinoids up to date but despite the large attention and focus on the possible medicinal benefits of cannabis derived compounds, there is still lack of candidate cannabinoids which may be used to effectively treat cancer.

Cannabidivarin (CBDV) for example, is a non-psychoactive cannabinoid found in Cannabis. CBDV has demonstrated anticonvulsant effects and GW Pharmaceuticals begun a phase 2 trial for adult epilepsy prompted by a demonstrated neurochemical pathway for previously observed anti-epileptic and anti-convulsive action.

International Patent Application Publication No. WO 2011/110866 discloses the use of phytocannabinoids in the treatment of colon cancer.

International Patent Application Publication No. WO 2008/129258 discloses the use of cannabinoid-containing plant extracts in the prevention or treatment of diseases or conditions, including colon cancer that are alleviated by blockade of one or more types of TRP channel.

Cannabis plants contain many compounds that can influence the plant's effect on humans. Several major cannabinoids in cannabis plant extracts represent up to 95% or more of the extract composition. On cells, those cannabinoids may produce a synergistic effect or might interfere with each other and/or possibly interact with other drugs like chemotherapies. In general, the interaction of medicaments can be divided into three major categories; antagonist, wherein one molecule inhibits the effects of another molecule, additive, wherein the combined medicaments produce an effect that equals the sum of either drug taken alone, and synergy, wherein a combination of molecules has a greater effect than the additive effect. Additive and synergistic antiproliferative effects may be favorable for the treatment and management of cancer as compared to administering either medicaments alone.

Nallathambi et al. (Cannabis and Cannabinoid Research 2018, 3, 120-135) disclose the identification of synergistic interaction between cannabis-derived compounds for cytotoxic activity in colorectal cancer cell lines and colon polyps that induces apoptosis-related cell death and distinct gene expression.

There is still a need for new and effective cannabinoid-based compositions that will provide a therapeutic alternative to treating cancers.

### SUMMARY OF THE INVENTION

The present invention pertains to compositions comprising cannabinoids for use in the treatment of cancer. Specifically, the present invention provides pharmaceutical compositions (herein collectively referred to as “cannabinoid composition” or “cannabinoid compositions”) comprising two or more cannabinoids selected from cannabichromenic acid (CBCA), cannabidivarin (CBDV), cannabidivarinic acid (CBDVA), tetrahydrocannabivarin (THCV) and cannabigerolic acid (CBGA). The herein cannabinoid compositions having at least two cannabinoids selected from the list of CBCA, CBDV, CBDVA, THCV, and CBGA are administered in an amount which exhibits a synergistic anti-cancer therapeutic effect.

The invention is based in part on the unexpected discovery that certain combinations of cannabinoids, specifically cannabinoid combinations selected from the list of: CBCA, CBDV, CBDVA, THCV, and CBGA, produce a superior cell proliferation inhibition/anti-cancer effect in in-vitro and in-vivo cancer models. While certain cannabinoids present no effect on cancer cells proliferation, when the herein cannabinoids are combined with other cannabinoids (all from the herein cannabinoid list), they present an enhanced anti-cancerous effect.

The present inventors therefore designed and successfully practiced novel anti-cancer pharmaceutical compositions that can be used in methods of treating cancer.

The cannabinoid combinations of the present invention provide synergistic effects i.e., the cannabinoids combinations provide a greater therapeutic result in treating cancer than the additive effects achieved by each individual constituent when administered at a therapeutic dose.

Thus, in a first aspect, the invention provides a composition for treating cancer comprising at least two cannabinoids selected from the group consisting of cannabichromenic acid (CBCA), cannabidivarin (CBDV), cannabidivarinic acid (CBDVA), tetrahydrocannabivarin (THCV) and cannabigerolic acid (CBGA).

In a further aspect, the invention provides a composition comprising at least two cannabinoids selected from the group consisting of CBCA, CBDV, CBDVA, THCV and CBGA, and a pharmaceutical acceptable carrier or excipient.

In some embodiments, the composition comprises two cannabinoids selected from the group consisting of CBCA, CBDV, CBDVA, THCV, and CBGA.

In some embodiments, the composition comprises three cannabinoids selected from the group consisting of CBCA, CBDV, CBDVA, THCV, and CBGA.

In some embodiments, the composition comprises four cannabinoids selected from the group consisting of CBCA, CBDV, CBDVA, THCV, and CBGA.

In some embodiments, the composition comprises the five cannabinoids CBCA, CBDV, CBDVA, THCV, and CBGA.

In some embodiments, the composition comprises at least two cannabinoids selected from the group consisting of CBGA and CBDV; CBGA and CBCA; CBGA and

CBDVA; CBGA and THCV; CBDV and CBCA; CBDV and CBDVA; CBDV and THCV; CBCA and CBDVA; CBCA and THCV; and CBDVA and THCV.

In some embodiments, the composition comprises CBDV and at least one additional cannabinoid selected from the group consisting of CBGA, CBCA, CBDVA, and THCV. In some embodiments, the composition comprises CBGA and at least one additional cannabinoid selected from the group consisting of CBDV, CBCA, CBDVA, and THCV. In some embodiments, the composition comprises CBCA and at least one additional cannabinoid selected from the group consisting of CBGA, CBDV, CBDVA, and THCV. In some embodiments, the composition comprises CBDVA and at least one additional cannabinoid selected from the group consisting of CBGA, CBCA, CBDV, and THCV. In some embodiments, the composition comprises THCV and at least one additional cannabinoid selected from the group consisting of CBGA, CBCA, CBDVA, and CBDV.

In some embodiments, the composition comprises at least CBDV and CBGA. In some embodiments, the % w/w ratio of CBDV and CBGA in the composition is between 1:99 and 99:1. In some embodiments, the % w/w ratio of CBDV and CBGA in the composition is between 40:60 to 60:40. In some embodiments, the % w/w ratio of CBDV and CBGA in the composition is between 1:10 and 10:1. In some embodiments, the % w/w ratio of CBDV and CBGA in the composition is 1:1.

In some embodiments, the composition further comprises THC. In some embodiments, the concentration of THC is between 0.1 to 10% w/w out of the total weight of THC, CBDV and CBGA. In some embodiments, the concentration of THC is 1 to 5% w/w out of the total weight of THC, CBDV and CBGA. In some embodiments, the concentration of THC is 2.5% w/w out of the total weight of THC, CBDV and CBGA. In some embodiments, the composition comprising CBDV/CBGA/THC at a % w/w ratio of 45:45:10 to 49:49:2 out of the total weight of THC, CBDV and CBGA. In some embodiments, composition comprising CBDV/CBGA/THC at a % w/w ratio of 48.75:48.75:2.5 out of the total weight of THC, CBDV and CBGA.

In some embodiments, the composition is substantially free of THC. In some embodiments, the composition is essentially free of THC. In some embodiments, the composition is free of THC.

In some embodiments, the composition further comprises CBD. In some embodiments, the composition is substantially free of CBD. In some embodiments, the composition is essentially free of CBD. In some embodiments, the composition is free of CBD.

In some embodiments, the composition comprises an immediate release formulation and a sustained-release formulation. In some embodiments, the immediate release formulation comprises an edible oil and the sustained release formulation comprises at least one Lipid-based Drug Delivery System (LBDDS) agent. In some embodiments, the LBDDS is selected from the group consisting of a monoglyceride, a diglyceride, a carrageenan and any mixture thereof. In some embodiments, the carrageenan is selected from the group consisting of lambda-carrageenan, kappa-carrageenan, iota-carrageenan and any mixture thereof. In some embodiments, the sustained release formulation further comprises an edible oil.

In some embodiments, the cannabinoid is selected from the group consisting of a synthetic cannabinoid, an isolated cannabinoid derived from a cannabis plant, a cannabis plant extract comprising at least 50% (w/w) of the herein cannabinoid.

In some embodiments, the cannabinoids in the composition exhibit a synergistic anti-cancer effect.

In some embodiments, the composition further comprises a chemotherapeutic agent. In some embodiments, the chemotherapeutic agent is an antimetabolite drug, a DNA alkylating agent, a platinum compound, an enzyme inhibitor (such as a Topoisomerase inhibitor, or a Tyrosine kinase inhibitor), a vincalkaloid, a taxane, a receptor antagonist, and an antibiotic.

In some embodiments, the antimetabolite drug is Capecitabine. In some embodiments, the taxane is Docetaxel. In some embodiments, the enzyme inhibitor is Epirubicin. In some embodiments, the antimetabolite drug is 5-Fluorouracil. In some embodiments, the antimetabolite drug is Capecitabin. In some embodiments, the platinum compound is Oxaliplatin. In some embodiments, the enzyme inhibitor is a Topoisomerase inhibitor. In some embodiments, the Topoisomerase inhibitors is Irinotecan.

In some embodiments, the composition is substantially free of a cannabinoid other than CBGA, CBDV, CBCA, CBDVA, and/or THCV. In some embodiments, the composition further comprises a carrier or excipient.

In yet another aspect, the present invention pertains to a method for treating cancer comprising administering to a subject in need a cannabinoid composition comprising at least two cannabinoids selected from the group consisting of CBCA, CBDV, CBDVA, THCV and CBGA.

In some embodiments, the composition is administered to the subject in need on a daily basis. In some embodiments, the dosage of the composition is increased gradually until reaching a maximum dosage. In some embodiments, the dosage of the composition is increased over a period of 1 week to 6 months. In some embodiments, the dosage of the composition is increased over a period of up to 3 months. In some embodiments, the composition is administered on a daily basis at least until disappearance of a tumor associated with the cancer. In some embodiments, the composition is administered on a daily basis for a period of between 4 months to 2 years. In some embodiments, the composition is administered on a daily basis for a period of 1 year.

In some embodiments, the treatment comprises inhibiting cancer cells' growth or proliferation, reducing or inhibiting the tumor volume, inducing cancer cells' death (via apoptosis and/or necrosis), or a combination thereof.

In some embodiments, the cancer is a solid cancer. In some embodiments, the solid cancer is selected from colorectal cancer, breast cancer and lung cancer.

In some embodiments, the method further comprises administering to the subject a chemotherapeutic agent. In some embodiments, the chemotherapeutic agent is administered concomitantly to, prior to or after administration of the cannabinoids composition.

In some embodiments, the composition is prescribed for oral administration.

In yet a further aspect the invention pertains to a composition comprising at least two cannabinoids selected from the group consisting of CBCA, CBDV, CBDVA, THCV and CBGA and a pharmaceutical acceptable carrier or excipient for use in the treatment of cancer.

In yet a further aspect the invention pertains to a composition comprising at least two cannabinoids selected from the group consisting of CBCA, CBDV, CBDVA, THCV and CBGA and a pharmaceutical acceptable carrier or excipient for the manufacture of a medicament for treating cancer. In some embodiments, the cannabinoid composition for preparation of a medicament for treating cancer.

In yet a further aspect, the invention provides a method for treating cancer comprising administering to a subject in need a therapeutic effective amount of the cannabinoid composition as defined herein.

In yet a further aspect, the invention provides a method of inhibiting cancer cells' proliferation or inducing cancer cells' death, comprising contacting the cancer cells with an effective amount of the cannabinoid composition as defined herein. In some embodiments, the cancer cells are colorectal cells, lung cancer cells or breast cancer cells.

The pharmaceutical composition of the present invention is particularly useful for treating or circumventing cancer. In some embodiments, the cancer is a solid tumor or a hematological malignancy. In some embodiments, the cancer is selected from the group consisting of colorectal cancer, lung cancer, breast cancer, melanoma cancer, stomach cancer, ovarian cancer, pancreatic cancer, head and neck cancer, squamous cell carcinoma, gastrointestinal cancer, breast cancer (such as carcinoma, ductal, lobular, and nipple), prostate cancer, non-small cell lung cancer, Non-Hodgkin's lymphoma, multiple myeloma, leukemia (such as acute lymphocytic leukemia, chronic lymphocytic leukemia, acute myelogenous leukemia, and chronic myelogenous leukemia), brain cancer, neuroblastoma, and sarcomas. In one or more embodiments, the cancer is colorectal cancer. In one or more embodiments, the cancer is a colon cancer. In one or more embodiments, the cancer is a lung cancer. In one or more embodiments, the cancer is a breast cancer.

### BRIEF DESCRIPTION OF THE DRAWINGS

For better understanding of the present invention and in order to exemplify how it may be implemented in practice, several embodiments are hereby described, which should be interpreted only as non-limiting examples, with reference to the accompanying figures. It is noted that the sizes and scale of the embodiments presented in the figures are exemplary and non-limiting.

It will be appreciated that for simplicity and clarity of illustration, where appropriate, reference numerals have been repeated among the different figures to indicate corresponding or analogous elements. In addition, numerous specific details are set forth in order to provide a thorough understanding of the embodiments described herein. However, it will be understood by those of ordinary skill in the art that the embodiments described herein can be practiced without these specific details. In other instances, methods, procedures, and components have not been described in detail so as not to obscure the related relevant feature being described. Also, the description is not to be considered as limiting the scope of the embodiments described herein.

FIGS. 1A-1B are bar graphs illustrating HT-29 colon cancer cells viability following exposure to each of CBDV and CBGA alone, or a combination thereof (FIG. 1A), or to each of CBGA and THC-d8 alone, or a combination thereof (FIG. 1B).

FIGS. 2A-2B are bar graphs illustrating A549 lung cancer cells viability following exposure to each of CBDV and CBGA alone, or a combination thereof (FIG. 2A), or to each of CBDVA and THC-d8 alone, or a combination thereof (FIG. 2B).

FIGS. 3A-3B are bar graphs illustrating MCF-7 breast cancer cells viability following exposure to each of CBDV and CBGA alone, or a combination thereof (FIG. 3A), or to each of CBDVA and THC-d8 alone, or a combination thereof (FIG. 3B).

FIGS. 4A-4B are bar graphs illustrating the viability and the synergistic effect of CBGA (at X1 concentration—25.6 μM; or X2 concentration—51.204) and CBDV (at X1 concentration—23.4 μM; or X2 concentration—46.8 μM) on HCT116 cells (FIG. 4A), or RKO cells (FIG. 4B).

FIGS. 5A-5B are bar graphs illustrating the viability and the synergistic effect of CBCA and THCV on cells of colon cancer biopsy (FIG. 5A) and the effect of CBG and CBDV on the viability of cells of colon cancer biopsy (FIG. 5B).

FIG. 6—is a bar graph illustrating the viability and the synergistic effect of CBGA (at X1 concentration—25.6 μM; and X2 concentration—51.204) and CBDV (at X1 concentration—23.5 μM; and X2 concentration—47 μM) on cells of human colon cancer biopsy.

FIGS. 7A-7B are bar graphs illustrating the viability following treatment with CBDV plus CBGA, CBDV plus CBGA plus Capecitabine, or Capecitabine alone, on HCT116 cells (FIG. 7A); or RKO cells (FIG. 7B).

FIGS. 8A-8B are bar graphs illustrating the viability of HCT116 cells following treatment with CBDV plus CBGA, or CBDV plus CBGA plus Docetaxel, or Docetaxel alone (FIG. 8A); or CBDV plus CBGA, or CBDV plus CBGA plus Irinotecan, or Irinotecan alone (FIG. 8B).

FIGS. 9A-9B are a tumor growth line graph (FIG. 9A) and a Kaplan-Meier survival analysis (FIG. 9B) of colon cancer bearing mice treated with a combination of CBDV and CBGA vs. vehicle as control.

### DESCRIPTION OF SPECIFIC EMBODIMENTS OF THE INVENTION

Although the invention is illustrated and described herein as embodied in examples provided herein, the invention is not limited to the details shown because various modifications and changes may be made without departing from the invention and the equivalents of the claims. However, the construction and method of operation of the invention together with additional objects and advantages thereof will be best understood from the following description of specific embodiments when read in connection with the accompanying figures.

The present invention pertains to novel cannabinoid compositions and to methods of using the same for treating cancer. The inventors of the present invention have surprisingly found that a combination of two cannabinoids selected from cannabichromenic acid (CBCA), cannabidivarin (CBDV), cannabidivarinic acid (CBDVA), tetrahydrocannabivarin (THCV) and cannabigerolic acid (CBGA) exhibits an anti-proliferative effect on various cancer cells being profoundly higher than the effect exhibited by the individual cannabinoids. The inventors have further surprisingly found that the combined treatment of the herein cannabinoid combinations with chemotherapy agents exhibits a synergistic anti-cancer therapeutic effect.

As used herein by Cannabidivarin (CBDV) it is meant to refer to the phytocannabinoid molecule 2-((1S,6S)-3-methyl-6-(prop-1-en-2-yl)cyclohex-2-enyl)-5-propylbenzene-1,3-diol, having the CAS number 24274-48-4.

As used herein by Cannabigerolic acid (CBGA) it is meant to refer to the acidic form (COOH) of cannabigerolic acid (3-[(2E)-3,7-dimethylocta-2,6-dienyl]-2,4-dihydroxy-6-pentylbenzoic acid) or its corresponding salts, having the CAS number 25555-57-1.

As used herein by Cannabichromenic acid (CBCA) it is meant to refer to a precursor of cannabichromen (CBC) biosynthesis, a non-psychoactive cannabinoid of Cannabis, having the formal name: 5-hydroxy-2-methyl-2-(4-methyl-3-penten-1-yl)-7-pentyl-2H-1-benzopyran-6-carboxylic acid and having the CAS number 185505-15-1.

As used herein by Cannabidivarinic acid (CBDVA) it is meant to refer to 2,4-dihydroxy-3-[(1R,6R)-3-methyl-6-(1-methylethenyl)-2-cyclohexen-1-yl]-6-propyl-benzoic acid. CBDVA is having the CAS number 31932-13-5

As used herein by Tetrahydrocannabivarin (THCV) it is meant to refer to 6aR,7,8,10aR-tetrahydro-6,6,9-trimethyl-3-propyl-6H-dibenzo[b, d] pyran-1-ol, having the CAS number 31262-37-0.

The scope of the invention also includes all different geometrical isomers (including cis, trans) of CBCA, CBDV, CBDVA, THCV and CBGA.

Thus, the present invention provides a pharmaceutical composition comprising at least two cannabinoids selected from the group consisting of CBCA, CBDV, CBDVA, THCV and CBGA. In some embodiments, the composition comprises at least three cannabinoids selected from the group consisting of CBCA, CBDV, CBDVA, THCV, and CBGA. In some embodiments, the composition comprises at least four cannabinoids selected from the group consisting of CBCA, CBDV, CBDVA, THCV, and CBGA. In some embodiments, the composition comprises CBCA, CBDV, CBDVA, THCV, and CBGA.

In some embodiments, the composition comprises two cannabinoids selected from the group consisting of CBCA, CBDV, CBDVA, THCV, and CBGA. In some embodiments, the composition comprises three cannabinoids selected from the group consisting of CBCA, CBDV, CBDVA, THCV, and CBGA. In some embodiments, the composition comprises four cannabinoids selected from the group consisting of CBCA, CBDV, CBDVA, THCV, and CBGA. In some embodiments, the composition comprises CBCA, CBDV, CBDVA, THCV, and CBGA.

In some embodiments, the composition consists essentially of two cannabinoids selected from the group consisting of CBCA, CBDV, CBDVA, THCV, and CBGA. In some embodiments, the composition consists essentially of three cannabinoids selected from the group consisting of CBCA, CBDV, CBDVA, THCV, and CBGA. In some embodiments, the composition consists essentially of four cannabinoids selected from the group consisting of CBCA, CBDV, CBDVA, THCV, and CBGA. In some embodiments, the composition consists essentially of CBCA, CBDV, CBDVA, THCV, and CBGA.

In some embodiments, the composition consists of two cannabinoids selected from the group consisting of CBCA, CBDV, CBDVA, THCV, and CBGA. In some embodiments, the composition consists of three cannabinoids selected from the group consisting of CBCA, CBDV, CBDVA, THCV, and CBGA. In some embodiments, the composition consists of four cannabinoids selected from the group consisting of CBCA, CBDV, CBDVA, THCV, and CBGA. In some embodiments, the composition consists of CBCA, CBDV, CBDVA, THCV, and CBGA.

In some embodiments, the herein cannabinoid compositions further comprise THC. In some embodiments, the composition comprises THC and a mixture of two or more cannabinoids selected from the group consisting of CBCA, CBDV, CBDVA, THCV and CBGA.

In some embodiments, the composition comprises CBCA, and CBDV. In some embodiments, the composition comprises CBCA and CBDVA. In some embodiments, the composition comprises, CBCA, and THCV. In some embodiments, the composition comprises CBCA, and CBGA. In some embodiments, the composition comprises CBDV and CBDVA. In some embodiments, the composition comprises CBDV and THCV. In some embodiments, the composition comprises CBDV and CBGA. In some embodiments, the composition comprises CBDVA, and THCV. In some embodiments, the composition comprises CBDVA, and CBGA. In some embodiments, the composition comprises THCV, and CBGA.

As illustrated in the examples section that follows, certain cannabinoids are further effective when provided as a sole active pharmaceutical ingredient. Thus, some aspects of the invention pertain to pharmaceutical compositions comprising, consisting essentially of, or consisting of at least one cannabinoid selected from the group consisting of CBCA, CBDV, CBDVA, THCV, and CBGA. In some embodiments, the invention pertains to methods of treating cancer comprising administering a pharmaceutical composition comprising, consisting essentially of, or consisting of at least one cannabinoid selected from the group consisting of CBCA, CBDV, CBDVA, THCV, and CBGA.

In some embodiments, the herein composition is substantially free, or essentially free, or entirely free of any other cannabinoid except for the one, two, or more of the cannabinoids selected from the group consisting of CBCA, CBDV, CBDVA, THCV, and CBGA. In some embodiments, the herein composition is substantially free or essentially free, or entirely free of any other cannabinoid except for THC and the one, two, or more of cannabinoids selected from the group consisting of CBCA, CBDV, CBDVA, THCV, and CBGA. In some embodiments, the composition is substantially free, or essentially free, or entirely free of any cannabinoids other than the one or two or more cannabinoids selected from the group listed above.

In some embodiments, the composition further comprises THC. One of the possible benefits of adding THC to the composition is to increase appetite and/or induce weight gain and/or muscle mass. In some embodiments, the composition further comprises CBD. In some embodiments, the composition comprises THC but excludes CBD. In some embodiments, the composition comprises CBD but excludes THC.

In some embodiments, the composition consists essentially of THC, CBDV and CBGA. In some embodiments, the composition comprises THC, CBDV and CBGA, and is substantially free of any other cannabinoid. In some embodiments, the composition is free of any other cannabinoid except for the herein CBCA, CBDV, CBDVA, THCV, and CBGA cannabinoids.

CBCA, CBDV, CBDVA, THCV, and CBGA can be obtained from a synthetic source or a natural plant source or from a cannabis plant extract.

A synthetic source means that the compound is synthesized by a chemical man-made process involving reacting a precursor or precursors in a chemical reaction to obtain a product in a single step or a multi-step synthesis.

A natural plant source refers to cannabinoids purified or isolated from a plant, typically extracted out from a mixture of compounds such as a mixture obtained by extraction of a plant source. The plant source may be a part of a plant, several parts of a plant or the whole plant. In some embodiments, the plant can be Cannabis. In some embodiments, the cannabis plant can be, for example, from the strains Cannabis sativa or Cannabis indica. A natural source can be a botanical drug substance such as an extract comprising a substantive amount of the target cannabinoid, for example, above about 90% w/w of the at least one cannabinoid selected from CBDV, CBGA, CBCA, CBDVA, or THCV (referred to herein also as “the designated cannabinoid”).

In embodiments comprising two cannabinoids selected from CBCA, CBDV, CBDVA, THCV and CBGA (e.g. CBDV and CBGA), the weight to weight ratio between the two cannabinoids may be between 1:99 to 99:1. For example, the weight ratio is between 1:90 to 90:1, between 1:80 to 80:1, between 1:70 to 70:1, between 1:60 to 60:1, between 1:50 to 50:1, between 1:40 to 40:1, between 1:30, to 30:1, between 1:20 to 20:1, between 1:10 to 10:1, between 10:90 to 90:10, between 1:8 to 8:1, between 1:7 to 7:1, between 1:6 to 6:1, between 1:5 to 5:1, between 1:4 to 4:1, between 1:3 to 3:1, between 1:2 to 2:1, or between 1:1.5 to 1.5:1. For example, the weight ratio is between 20:80 to 80:20, in some embodiments between 30:70 to 70:30 and in some embodiments between 40:60 to 60:40, respectively. In some embodiments, the weight-to-weight ratio between the two cannabinoid ingredients is 1:1 to 1:10, or 1:1 to 1:9, or 1:1 to 1:8, or 1:1 to 1:7, or 1:1 to 1:6, or 1:1 to 1:5, or 1:1 to 1:4, or 1:1 to 1:3, or 1:1 to 1:2, or any value in between. In some embodiments, the weight-to-weight ratio between the two cannabinoid ingredients is 1:1. In some embodiments, the weight-to-weight ratio between CBDV and CBGA is 1:1.5 to 1.5:1, or any value in between.

The molar ratio between the two cannabinoids that are selected from CBCA, CBDV, CBDVA, THCV and CBGA (e.g. CBDV and CBGA) may be between 1:99 to 99:1. For example, the molar ratio is between 1:90 to 90:1, between 1:80 to 80:1, between 1:70 to 70:1, between 1:60 to 60:1, between 1:50 to 50:1, between 1:40 to 40:1, between 1:30, to 30:1, between 1:20 to 20:1, between 1:10 to 10:1, between 10:90 to 90:10, between 1:8 to 8:1, between 1:7 to 7:1, between 1:6 to 6:1, between 1:5 to 5:1, between 1:4 to 4:1, between 1:3 to 3:1, between 1:2 to 2:1, or between 1:1.5 to 1.5:1, or any value in between. In some embodiments, the molar ratio is between 20:80 to 80:20, in some embodiments between 30:70 to 70:30 and in some embodiments between 40:60 to 60:40, respectively. In some embodiments, the molar ratio between the two cannabinoid ingredients is 1:1 to 1:10, or 1:1 to 1:9, or 1:1 to 1:8, or 1:1 to 1:7, or 1:1 to 1:6, or 1:1 to 1:5, or 1:1 to 1:4, or 1:1 to 1:3, or 1:1 to 1:2, or any value in between. In some embodiments, molar ratio between the two cannabinoid ingredients is 1:1. In some embodiments, the molar ratio between CBGA and CBDV is between 1:10 to 10:1, or between 1:3.5 to 8.5:1, or between 1:1.5 to 1.5:1. In some embodiments, the molar ratio between CBGA and CBDV is 1:1.25, or any value in between.

In some embodiments, the cannabinoid is present in the composition at a concentration of up to about 1000 μM. For example, up to about 500 μM, up to about 400 μM, up to about 300 μM, up to about 200 μM, or up to about 100 μM. For example, the cannabinoid may be present in the composition at a concentration of between about 2 μM and about 100 μM, between about 5 μM and about 100 μM, between about 5 μM and about 90 μM, between 5 μM and 80 μM, between 5 μM and 70 μM, between about 5 μM and about 60 μM, or between 10 μM and about 55 μM, or any value in between.

In some embodiments, the cannabinoid is present in the composition at a concentration of between about 5 mM and about 1000 mM, between about 5 mM and about 500 mM. between about 5 mM and about 100 mM. For example, between about 5 mM and about 50 mM, between 5 mM and 40 mM, between 5 mM and about 30 mM, between about 5 mM and about 20 mM, or between 10 mM and about 20 mM, or any value in between, or any value in between.

In some embodiments, the CBDV is present in the composition at a concentration of between about 5 mM and about 1000 mM, between about 5 mM and about 500 mM. Between about 5 mM and about 100 mM. For example, between about 5 mM and about 50 mM, between 5 mM and 40 mM, between 5 mM and about 30 mM, between about 5 mM and about 20 mM, or between 10 mM and about 20 mM, or any value in between. In an exemplary embodiment, the CBDV is present in the composition at a concentration of 17.46 mM.

In some embodiments, the CBGA is present in the composition at a concentration of between about 5 mM and about 1000 mM, between about 5 mM and about 500 mM, between about 5 mM and about 100 mM. For example, between about 5 mM and about 50 mM, between 5 mM and 40 mM, between 5 mM and about 30 mM, between about 5 mM and about 20 mM, or between 10 mM and about 20 mM. In an exemplary embodiment, the CBGA is present in the composition at a concentration of 13.87 mM.

In some embodiment, the weight to volume of each cannabinoid in the composition is between 1% w/v to 80% w/v. For example, 1% w/v/ to 50% w/v, 1% w/v/ to 40% w/v, 1% w/v/ to 30% w/v, 1% w/v/ to 20% w/v, or 5% w/v/ to 10% w/v, or any value in between. In an exemplary embodiment, the weight to volume of each cannabinoid is 10% w/v.

In some embodiments, the herein cannabinoid compositions may comprise THC in a relatively small amount as compared to the amount of the one or at least two other cannabinoids in the composition. In some embodiments, the concentration of THC is between 0.1 to 10% w/w, 1 to 5% w/w, or 2.5% w/w out of the total weight of THC and the one, or two or more other cannabinoids (e.g., CBDV and CBGA).

In some embodiments, the composition comprises CBDV/CBGA/THC at a weight-to-weight ratio of 45:45:10 (the ratio is the weight ratio out of the total weight of CBDV, CBGA and THC). In some embodiments, the composition comprises CBDV/CBGA/THC at a weight-to-weight ratio of 48.75:48.75:2.5. In some embodiments, it comprises a weight-to-weight ratio of 45:45:10 to 49:49:2% w/w out of the total weight of THC, CBDV and CBGA. For example, the composition may contain 100 mg CBDV, 100 mg CBGA and 2.5 mg THC.

The total amount of cannabinoids selected from CBCA, CBDV, CBDVA, THCV and CBGA in a dosage unit form can be up to about 2000 mg, For example, between about 10 mg and about 2000 mg. For example, the total amount can be about 100 mg, 200 mg, 300 mg, 400 mg, 500 mg, 600 mg, 700 mg, 800 mg, 900 mg, 1000 mg, 1200 mg, 1500 mg, or 2000 mg. For example, the total amount of cannabinoids can be between about 10 mg and 1500 mg, between about 10 mg and 1000 mg, between about 10 mg and about 800 mg, between about 10 mg and 500 mg, between about 100 mg and 800 mg, or between about 100 mg and 500 mg, or any value in between.

The amount of THC in the composition can be between 0.1 mg to 30 mg per dosage unit form. In some embodiments, the amount of THC in the composition can be 0.1, 0.5, 1, 2, 3, 4, 5, 6, 7, 8, 9, 10, 15 or 15 mg. The amount of CBD in the composition can be between 0.05 mg to 5 mg per dosage unit form. In some embodiments, the amount of CBS in the composition can be 0.1, 0.15, 0.20, 0.25, 0.50, 1.0 or 1.5 mg, or any value in between.

In some embodiment, both THC and CBD are present in the composition and the total amount of THC and CBD in the composition is between 1 to 10 mg. In some embodiments, THC and CBD are present in the composition in a relative ratio of THC/CBD 95:5 wt % out of the total weight of THC and CBD.

In some embodiments, the composition exhibits a synergistic effect of the two or more cannabinoids selected from the list provided above. In some embodiments, the composition exhibits a synergistic effect of CBDV and CBGA.

The herein composition comprising one or more cannabinoids selected from the group consisting of CBCA, CBDV, CBDVA, THCV and CBGA may be prescribed for administration or provided along with the treatment of a chemotherapeutic agent. The composition comprising one or more cannabinoids selected from the group consisting of CBCA, CBDV, CBDVA, THCV and CBGA can be administered before, concomitantly to or after the administration of a chemotherapeutic agent. Optionally, the herein cannabinoid compositions comprising cannabinoids selected from the group consisting of CBCA, CBDV, CBDVA, THCV and CBGA may additionally include a chemotherapeutic agent.

As used herein “chemotherapeutic agents” are drug treatments that uses chemicals to eradicate fast-growing cells. As used herein the chemotherapeutic agent is not a cannabinoid. Suitable chemotherapeutic agents include, but are not limited to, antimetabolite drugs, DNA alkylating agents, platinum compounds, enzyme inhibitors (e.g., topoisomerase inhibitors, tyrosine kinase inhibitors), vincalkaloids (vinka-alkaloid), taxanes (e.g., paclitaxel, docetaxel, and cabazitaxel) receptor antagonists, and antibiotics.

The invention also pertains to chemotherapeutic combination therapies, including by not limited to the combinations FOLFOX (folinic acid (leucovorin), fluorouracil (5-FU) and Oxaliplatin) and FOLFIRI (folinic acid (leucovorin), fluorouracil (5-FU) and irinotecan).

A “DNA alkylating agent” is an agent which attaches an alkyl group to DNA and thereby prevents its replication. Non-limiting examples of DNA alkylating agents are Nitrogen mustards, such as Cyclophosphamide, Mechlorethamine, Uramustine, Melphalan, Chlorambucil, Ifosfamide, Bendamustine; Nitrosoureas, such as Carmustine, Lomustine, Streptozocin; Alkyl sulfonates, such as Busulfan; and ThioTEPA.

“Platinum compounds” are a subclass of the DNA alkylating agents. Non-limiting examples of such compounds include Cisplatin, Carboplatin, Nedaplatin, Oxaliplatin, Satraplatin, and Triplatin tetranitrate.

“Topoisomerase inhibitors” are agents that interfere with the action of topoisomerase enzymes (topoisomerase I and II). Non-limiting examples of Topoisomerase I inhibitors include CPT-11/Irinotecan, topotecan, camptothecin, and lamellarin D. Non-limiting examples of Topoisomerase II inhibitors include, but are not limited to, Etoposide, Teniposide, Anthracyclines (e.g., Doxorubicin, Daunorubicin, Epirubicin), Mitoxantrone, amsacrine, aurintricarboxylic acid, and ellipticines.

“Tyrosine kinase inhibitors” (TKIs) are a class of chemotherapy medications that inhibit, or block, the enzyme tyrosine kinase. Non limiting examples of TKI chemotherapeutic agents include: Imatinib (brand name: Gleevac), gefinitib (Iressa) Erlotinib (Tarceva), Lapatinib (Tykerb), Sunitinib (Sutent), Sorafenib (Nexavar), Nilotinib (Tasinga), Bosutinib, Neratinib, and Vatalanib.

“Antimetabolite drugs” include pyrimidine antagonists, purine antagonists and folic acid analogues. Non limiting examples of pyrimidine antagonists include: 5-fluoruracil (5-FU, 5FU), arabinosylcytosine (cytarabine), capecitabine (an oral 5-FU pro-drug), gemcitabine and decitabine.

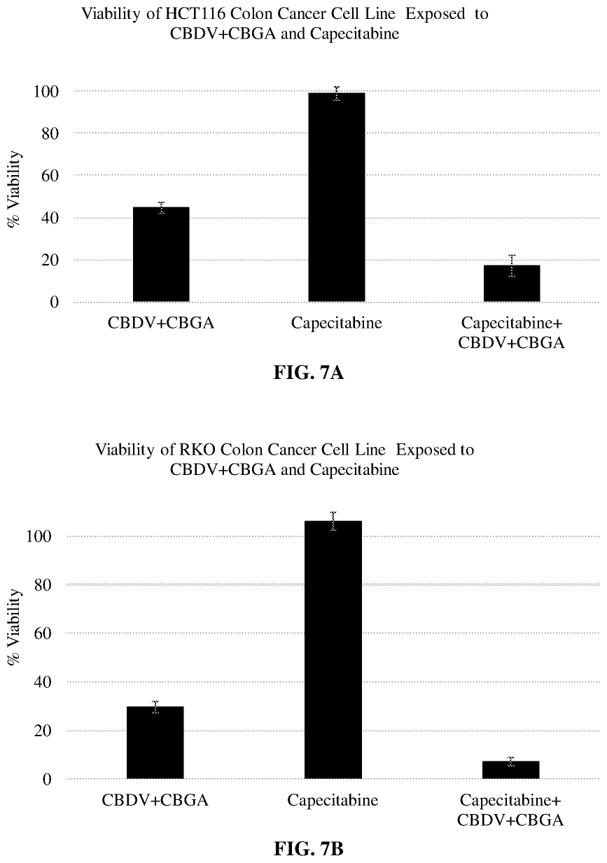

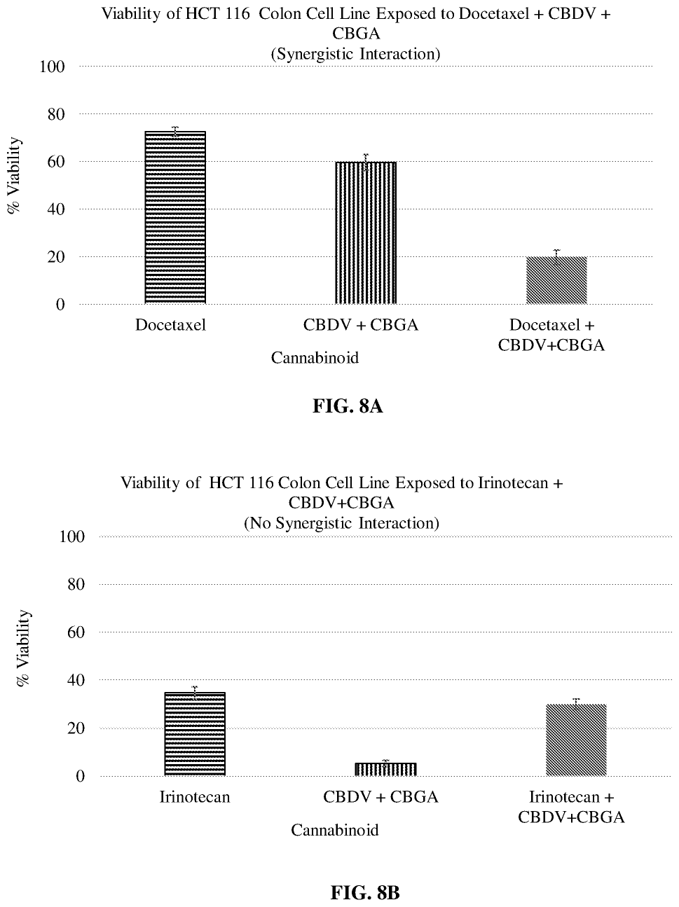

Cancer patients often receive chemotherapy and at the same time, use cannabinoids for relief of different symptoms associated with cancer or adverse effects associated with the chemotherapy treatment. Nevertheless, simultaneous administration of various active pharmaceutical agents may lead to pharmaceutical drug interference. Alternatively, APIs administered concomitantly may illustrate an additive reaction or a synergistic reaction that will increase the toxicity of the chemotherapeutic drug. As illustrated in the examples section that follows, and in FIGS. 7A-7B, and 8A, the combined anti-cancer effect of Capecitabine with CBDV and CBGA is synergistic (FIGS. 7A-7B). Further, the combined anti-cancer effect of additional chemotherapy agents, such as Docetaxel, with CBDV and CBGA is synergistic (FIG. 8A).

Thus, in some embodiments, the herein cannabinoid composition comprises one or more cannabinoid selected from CBCA, CBDV, CBDVA, THCV and CBGA, and one or more chemotherapeutic agents.

In an exemplary embodiment, the chemotherapeutic agent is an antimetabolite drug. As used herein the term “antimetabolite drugs” includes pyrimidine antagonists, purine antagonists and folic acid analogues. Non limiting examples of pyrimidine antagonists include: 5-fluoruracil (5-FU, 5FU), arabinosylcytosine (cytarabine), capecitabine (an oral 5-FU pro-drug), gemcitabine and decitabine.

In some embodiments, the weight to weight ratio between the cannabinoid(s) and the chemotherapeutic agent is between 1:99 to 99:1. In some embodiments, the weight ratio is between 1:90 to 90:1, between 1:80 to 80:1, between 1:70 to 70:1, between 1:60 to 60:1, between 1:50 to 50:1, between 1:40 to 40:1, between 1:30, to 30:1, between 1:20 to 20:1, between 1:10 to 10:1, between 1:9 to 9:1, between 1:8 to 8:1, between 1:7 to 7:1, between 1:6 to 6:1, between 1:5 to 5:1, between 1:4 to 4:1, between 1:3 to 3:1, between 1:2 to 2:1, or between 1:1.5 to 1.5:1, or any value in between. In some embodiments, the weight ratio is between 3:7 to 7:3 and in some embodiments between 2:3 to 3:2, respectively. In some embodiments, the ratio between the two cannabinoid ingredients and the chemotherapeutic agent is 1:1 to 1:10, or 1:1 to 1:9, or 1:1 to 1:8, or 1:1 to 1:7, or 1:1 to 1:6, or 1:1 to 1:5, or 1:1 to 1:4, or 1:1 to 1:3, or 1:1 to 1:2, or any value in between. In some embodiments the weight-to-weight ratio between the cannabinoid(s) and chemotherapeutic agent is 1:1. In some embodiments, the weight-to-weight ratio is between 1:1.5 to 1.5:1.

In some embodiments, the molar ratio between the cannabinoid(s) and the chemotherapeutic agent is between 1:99 to 99:1. In some embodiments, the molar ratio is between 1:90 to 90:1, between 1:80 to 80:1, between 1:70 to 70:1, between 1:60 to 60:1, between 1:50 to 50:1, between 1:40 to 40:1, between 1:30, to 30:1, between 1:20 to 20:1, between 1:10 to 10:1, between 1:9 to 9:1, between 1:8 to 8:1, between 1:7 to 7:1, between 1:6 to 6:1, between 1:5 to 5:1, between 1:4 to 4:1, between 1:3 to 3:1, between 1:2 to 2:1, or between 1:1.5 to 1.5:1, or any value in between. In some embodiments, the molar ratio is between 3:7 to 7:3 and in some embodiments between 2:3 to 3:2, respectively. In some embodiments the molar ratio between the cannabinoid(s) and chemotherapeutic agent is 1:1. In some embodiments, the molar ratio is between 1:1.5 to 1.5:1. In some embodiments, the molar ratio is 1:1 to 1:10, or 1:1 to 1:5.

### Formulation of the Composition

In some embodiments, the herein cannabinoid compositions, containing one or more of CBCA, CBDV, CBDVA, THCV and CBGA, may be formulated as an oral composition. Oral compositions generally include incorporating the mixture of cannabinoid(s) with an edible carrier or an inert diluent.

Various alternative administration routes are further applicable and herein contemplated including, intravenous administration (i.v.), sublingual administration, intraperitoneal administration (i.p.), intranasal administration for a direct pulmonary dosing, a rectal administration, intramuscular administration, subcutaneous administration, transdermal administration, and/or intraocular administration.

The composition can be formulated as granules, powder, capsules, tablet, film, emulsion, lozenge, tincture, sachets, a chewing gum, a hard or soft gelatin capsule or a suspension. In some embodiments, the oral composition is enclosed in a soft gelatin capsule. In other embodiments, the composition is compressed into tablets. In some embodiments, the oral composition is formulated as a mouth wash by using one or more diluents in which the cannabinoid or a mixture thereof is dissolved, suspended or emulsified in the fluid carrier.

Exemplary diluents are water and alcohols, for example, ethanol, benzyl alcohol and the polyethylene alcohols, either with or without the addition of a pharmaceutically acceptable surfactant, suspending agent, or emulsifying agent. The mouth wash is applied orally to the buccal cavity and it may then be swished and expectorated or swallowed. Pharmaceutically compatible binding agents, and/or adjuvant materials can be included as part of the composition. Capsules may contain surfactants, lubricants, and inert fillers, such as lactose, sucrose, calcium phosphate, and corn starch. Tablet forms may include one or more of lactose, sucrose, mannitol, corn starch, potato starch. alginic acid, microcrystalline cellulose, acacia, gelatin, guar gum, colloidal silicon dioxide, talc, magnesium stearate, calcium stearate, zinc stearate, stearic acid, and other excipients, colorants, diluents, buffering agents, disintegrating agents, moistening agents, preservatives, flavoring agents, and pharmacologically compatible carriers. Lozenges can comprise the active ingredient in a flavor, usually sucrose and acacia or tragacanth, as well as pastilles comprising the active formulation in an inert base, such as gelatin and glycerin, or sucrose and acacia, emulsions, gels, and the like containing, in addition to the active formulation, such carriers as are known in the art.

In some embodiments, the composition can be formulated in a slow-release form, an immediate release form or a combination thereof. As an example, the immediate release formulation may comprise an edible oil and the sustained release formulation may comprise at least one Lipid-based Drug Delivery System (LBDDS) agent. In some embodiments, the LBDDS agent is selected from the group consisting of a monoglyceride, a diglyceride, a carrageenan and any mixture thereof. In some embodiments, the carrageenan is selected from the group consisting of lambda-carrageenan, kappa-carrageenan, iota-carrageenan and any mixture of the carrageenan.

In some embodiments, the composition comprises carrageenan in a relative amount of about 0.005 V/V out of the total volume of the composition.

In some embodiments, the sustained release formulation further comprises an edible oil such as coconut oil, wheat sprout oil, olive oil, sprouted wheat oil, sesame oil, peanut oil, grape seed oil, palm oil, papaya seed oil or any combination thereof.

In some embodiments, the composition may further comprise cannabis extract in a relative amount range of about 0.01 V/V to about 0.02 V/V out of the total volume of the composition and the cannabinoid(s) comprises between 10 to 200 of mg of each one of CBDV and CBGA, CBCA, CBDVA, and THCV.

In some embodiments, the composition comprises mono- and diglyceride in a relative amount of about 0.075 V/V. In some embodiments, the composition comprises coconut oil in a relative amount of about 0.92 V/V.

In some embodiments the composition further comprises at least one excipient selected from the group consisting of a diluent, a binder, a lubricant, a disintegrant, a flavoring agent, a coloring agent, a stabilizer, a surfactant, a glidant, a plasticizer, a preservative, an essential oil and a sweetener.

In some embodiments, the therapeutic effect of the composition has direct immediate effect and/or a duration of up to 24 hours or from 4 to 24 hours.

In some embodiments, the composition comprising cannabinoid(s) (e.g., cannabis extract, isolated cannabinoid, synthetic cannabinoid) in a relative amount of about 0.01 V/V, Carrageenan in a relative amount of about 0.005 V/V, Mono and Diglyceride in a relative amount of about 0.075 V/V and coconut oil in a relative amount of about 0.92 V/V out of the total volume of the composition.

In some embodiments, both the immediate release portion and the slow-release portion of the composition comprises a homogenous mixture of all the cannabinoids and optionally chemotherapeutic agents of the composition.

In some embodiments, the immediate release portion of the composition comprises THC, and the slow-release portion comprises the two other cannabinoids of the composition. For example, the composition may contain THC in the immediate release formulation and a mixture of CBDV and CBGA in the slow-release formulation. Such an immediate release slow-release composition can achieve two purposes—a palliative effect by the THC ingredient and a slow-release anti-cancer effect by the two other cannabinoids. The THC which is released from the immediate release component can be present in a lower concentration than the two cannabinoid mixture in the slow-release component of the composition.

### Method of Treatment

In a further aspect, the invention provides a method for treating a subject suffering from cancer comprising administering to the subject in need a therapeutically effective amount of the cannabinoid composition of this invention.

In a further aspect, the invention provides a method for inhibiting cancer cells' growth or cancer cells' proliferation, or reducing or inhibiting cancer cells' viability, or inducing cancer cells apoptosis and/or necrosis, the method comprising contacting cancer cells with an effective amount of the cannabinoid composition of this invention.

In a further aspect, the invention provides a method for treating a subject suffering from cancer comprising administering to the subject in need a therapeutically effective amount of the cannabinoids and a chemotherapeutic agent composition of this invention.

In a further aspect, the invention provides a method for inhibiting cancer cells' growth or cancer cells' proliferation, or inducing cancer cells apoptosis and/or necrosis, the method comprising contacting cancer cells with an effective amount of the herein cannabinoid composition and chemotherapeutic agent.

In yet a further aspect, the invention provides a pharmaceutical composition comprising a therapeutic effective amount of the cannabinoid composition of this invention for use in the treatment of cancer.

The term “treating” or “treatment” of cancer in a patient refers to administration of the herein cannabinoid composition for preventing one or more of the cancer symptoms, inhibiting cancer progression, stabilizing its progression, reducing the volume of the tumor, or ameliorating or delaying the appearance of one or more of its symptoms.

The term “subject” refers to a human or a non-human mammal.

The term “administration” refers to providing or giving a subject a therapeutic agent by any effective route.

The term “effective amount” or “therapeutically effective amount” refers to a quantity sufficient to, when administered to the subject, affect a beneficial or desired result, including clinical results such as inhibiting cellular growth/proliferation, such as tumor or cancer cells' growth (or both), tumor volume, or a combination thereof.

In some embodiments, the method for treating cancer includes administrating to the subject in need a dosage of a therapeutically effective amount of the composition. Optionally, the dose for administration is a daily administration dose. The therapeutically effective amount dosage to be administered and the particular mode of administration will vary depending upon such factors as the age, weight of the particular subject, the therapeutic or diagnostic use contemplated, and the form of the formulation. In some embodiments, the administration of a therapeutically effective amount of the composition is preceded by a build-up period in which less than the target therapeutically effective amount is administered daily to the patient and is gradually increase until reaching the therapeutic effective amount of the composition to the patient. This allows the patient to gradually accustom to the composition, which may among other benefits, allow better compliance of the subject to the treatment. The buildup period may last for several days to several weeks. In some embodiments, the buildup period can last 5, 10 or 15 days. In some embodiments, the buildup period can take 2, 3, 4, 5, 6, 7, 8, 9 or 10 weeks. For example, the treatment may start with administering a single daily dosage to the subject in need during the first week of treatment, then increasing the regimen to two daily dosages in the second week, three daily dosages during the third week and reaching the target four daily dosages in the fourth week.

The single daily dosage may, for example, comprise 100 mg of CBDV, 100 mg of CBGA and optionally also 5 mg of THC, to be taken once a day during the first week, twice a day (for example every 12 hours) during the second week, three times a daily (for example every 8 hours) during the third week and every 6 hours during the fourth week onwards. The dosage units can be taken separately as exemplified above or together, either as several dosages taken together at the same occasion daily or by administering a single dosage having an increased amount of active ingredients (i.e., a capsule having 100 mg of CBDV, 100 mg of CBGA and optionally 5 mg of THC during the first week, a capsule having 200 mg of CBDV, 200 mg of CBGA and optionally 10 mg of THC during the second week, and so forth).

In some embodiment, the herein cannabinoid composition is administered concomitant to, after, or before the administration of a chemotherapeutic agent. In some embodiments, treatment includes cannabinoid(s) administration wherein the cannabinoids are provided as separate unit dosage forms.

The treatment period (starting from the first day of the buildup period) can last for as long as the condition of the subject in need requires, i.e., until the offset of symptoms of the disease such as the disappearance of the tumor as may be indicated by imaging techniques, or blood levels of markers or combination thereof. In some embodiments the duration of the treatment is about four months. In some embodiments, the treatment duration is 6, 8, 10, 12 or 18 months.

In another aspect, the invention provides the use of the composition according to the invention in the manufacture of a medicament for treating cancer. In another aspect, the invention provides the use of the composition of the invention for the treatment of cancer.

The cancer amendable for treatment by the present invention include solid tumor or a hematological tumor. The cancers for treatment include, but are not limited to, carcinoma, lymphoma, blastoma, sarcoma, and leukemia or lymphoid malignancies. More particular examples of such cancers include squamous cell cancer, lung cancer (including small-cell lung cancer, non-small cell lung cancer, adenocarcinoma of the lung, and squamous carcinoma of the lung), skin cancer (e.g., melanoma), cancer of the peritoneum, hepatocellular cancer, gastric or stomach cancer (including gastrointestinal cancer), pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney or renal cancer, liver cancer, prostate cancer, vulval cancer, thyroid cancer, hepatic carcinoma and various types of head and neck cancer, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); Hairy cell leukemia; chronic myeloblastic leukemia; and post-transplant lymphoproliferative disorder (PTLD), as well as abnormal vascular proliferation associated with phakomatoses, edema (such as that associated with brain tumors), and Meigs' syndrome. Preferably, the cancer is selected from the group consisting of colon, kidney, adrenocortical and hepatocellular cancers; breast cancer, Acute Myelogenous Leukemia (AML), Chronic lymphocitic leukemia (CLL), pro-lymphocitic leukemia, oesophagal carcinoma, non-small-cell lung cancers, soft-tissue sarcomas and osteosarcomas.

In an exemplary embodiment, the cancer is colorectal cancer. By “colorectal cancer” it is meant to refer to both colon cancer and rectal cancer.

The herein pharmaceutical composition may be administered orally, parenterally, by inhalation or spray, sublingually, transdermally, via buccal administration, rectally, as an ophthalmic solution, intravenously, intraperitoneally, topically, or by other means, in dosage unit formulations containing conventional pharmaceutically acceptable carriers.

The pharmaceutical composition may be formulated as any pharmaceutically useful form, e.g., as an aerosol, a cream, a gel, a pill, a capsule, a tablet, a syrup, a transdermal patch, or an ophthalmic solution. Some dosage forms, such as tablets and capsules, are subdivided into suitably sized unit doses containing appropriate quantities of the active components, e.g., an effective amount to achieve the desired purpose.

### Definitions

As used herein the term “Phytocannabinoids” are cannabinoids that originate from nature and can be found in the cannabis plant. The phytocannabinoids can be isolated from plants to produce a highly purified extract or can be reproduced synthetically. Phytocannabinoids can be obtained as either the neutral (decarboxylated form) or the carboxylic acid form depending on the method used to extract the cannabinoids. For example, it is known that heating the carboxylic acid form will cause most of the carboxylic acid form to decarboxylate into the neutral form.

As used herein the term “synthetic compound” refers to a chemical man-made compound/molecule functionally and structurally similar to its corresponding natural compound. Synthetic compounds are not derived from plants. Synthetic compounds bind to same receptors to which the natural corresponding plant compounds attach. These synthetic analogs often, but not necessarily, have greater binding affinity and greater potency to the respective receptor. The synthetic compound herein refers to a cannabinoid selected from CBCA, CBDV, CBDVA, THCV, and CBGA (hereinafter “the designated cannabinoid”), and optionally THC.

As used herein, the terms “isolated cannabinoid”, “purified cannabinoid” and “Highly purified cannabinoid” are interchangeable and refer to a compound (herein “the designated cannabinoid”) which is extracted from the cannabis plant and purified to the extent that other cannabinoids and non-cannabinoid components that are co-extracted with the cannabinoids have been removed, such that the highly purified cannabinoid is greater than or equal to 95% (w/w) pure. In this context “isolated” can be taken to mean that no substantial or essential amounts of components with which the designated cannabinoid is usually associated in nature or in in vitro conditions other than the designated compound and optionally its carrier or solvent is detectable by high pressure liquid chromatography (HPLC).

In one or more embodiments, the isolated compound is substantially free of any compounds other than the designated cannabinoid.

In one or more embodiment, the isolated cannabinoid is essentially free of any compounds other than the designated compound.

In one or more embodiment, the isolated cannabinoid is free of compounds other than the designated cannabinoid.

As used here, the term “substantially free” refers to a compound provided in a highly purified/isolated form such that it comprises less than about 0.5%, or less than about 0.4%, or less than about 0.3%, or less than about 0.2%, or less than about 0.1%, or any percentage in between of compounds other than the designated cannabinoid.

As used herein, the term “essentially free” refers to a compound provided in a highly purified/isolated form such that it comprises less than about 0.05%, or less than about 0.04%, or less than about 0.03%, or less than about 0.02%, or less than about 0.01%, or less than about 0.005%, or any percentage in between, or having only trace amounts of compounds other than the designated cannabinoid.

The term “extract” refers hereinafter to any fraction or concentrate derived from a plant which contains at least one of the herein designated cannabinoids. A natural phytocannabinoid may be extracted and/or fractioned from its respective cannabis plant using any one of the many known methods, such as nonhydrocarbons extraction methods (e.g., carbon dioxide extraction) and hydrocarbons extraction methods (e.g., butane, propane or alcohol extractions). It further refers to extracts treated by separation or purification or fractionation processes.

The extract or fraction may be enriched with the designated cannabinoid, namely, the designated cannabinoid with which the extract is enriched is present in an amount that is higher than that of the other compounds. Optionally, the designated cannabinoid is present in the extract in an amount greater than 40% (w/w) of the total extract. For example, the designated cannabinoid is present in an amount greater than 50% (w/w), greater than 60% (w/w), greater than 75%, greater than 80%, greater than 85%, or greater than 90% of the extract. A fraction or extract typically includes other compounds or ingredients of the extracted or fractioned plant. In some embodiments, the fraction or extract includes at least about 0.5% of the plant derived compounds other than the designated cannabinoid. In some cases, fractions that contain certain amounts of additional compounds, other than the designated cannabinoid, allow for an enhanced efficacy due to the entourage effect of the combination of compounds.

Optionally, a composition, especially composition comprising a cannabis plant derived molecule includes one or more of terpene and/or terpenoid. Alternatively, a composition comprising a cannabis plant derived molecule does not include any terpenes and/or terpenoids. The terpene/terpenoid may be provided within a cannabis extract or fraction.

The term “terpenes” or “terpenoids” as used herein refers to a class of hydrocarbon molecules, which often provide a unique smell. Terpenes are derived from units of isoprene, which has the molecular formula C₅H₈. The basic molecular formula of terpenes are multiples of the isoprene unit, i.e. (C₅H₈)ₙ where n is the number of linked isoprene units. Terpenoids are terpene compounds that have been further metabolized in the plant, typically through an oxidative process, and therefore usually contain at least one oxygen atom.

The term “consisting essentially of” means that the composition, method or structure may include additional ingredients, steps and/or parts, but only if the additional ingredients, steps and/or parts do not materially alter the basic and novel characteristics of the claimed composition, method or structure.

The term “consisting of” means including and limited to.

As used herein the term “about” refers to ±10% or ±5%.

The terms “comprise”, “comprising”, “includes”, “including”, “having” and their conjugates mean “including but not limited to”.

As used herein the term “synergistic” refers to e.g., an observed effect which is higher in the presence of the cannabinoids together than the sum of the individual effects of each cannabinoid when administered separately. In one embodiment, the observed combined effect of the cannabinoids is significantly higher than the sum of the individual effects. The term significant means that the statistical p value observed is less than 0.05, i.e., p<0.05. It is contemplated that the therapeutic effects achieved as a result of the combination of two or more cannabinoids selected from CBCA, CBDV, CBDVA, THCV and CBGA are significantly better than the expected additive therapeutic effects. Thus, the doses of each of the agents can be reduced.

In some embodiments, the synergistic effect can be calculated according to the cells mortality experimental results according to the Bliss equation as follows:

Mortality MAB(Cal)=MA+MB−MA*MB

Synergy (>0)=MAB (Exp)−MAB (Cal)

M designates mortality; wherein MAB—is a calculation of mortality, e.g., of cells exposed to a combination of cannabinoid A and cannabinoid B; MA is the mortality of cells exposed to cannabinoid A only; MB is the mortality of cells exposed to cannabinoid B only. MAB designates the observed mortality of cells exposed to the combination of cannabinoid A and cannabinoid B.

Alternatively, the synergistic effect can be calculated according to the cells survival experimental results according to the Bliss equation as follows:

Survival SAB(Cal)=SA×SB

Synergy(>0)=SAB(Cal)−SAB(Exp)

S designates mortality; wherein SAB (Cal)—is a calculation of survival, e.g., of cells exposed to a combination of cannabinoid A and cannabinoid B; SA is the survival of cells exposed to cannabinoid A only; SB is the survival of cells exposed to cannabinoid B only. SAB (Exp) designates the observed survival of cells exposed to the combination of cannabinoid A and cannabinoid B.

### EXAMPLES

The invention will be described in detail by way of specific examples. The following examples are offered for illustrative purposes and are not intended to limit the invention in any manner. Those of skill in the art will readily recognize a variety of non-critical parameters, which can be changed or modified to yield essentially the same results.

In one or more embodiments, the amounts in the examples should be read with the prefix “about”.

### Materials

Cannabinoids—the cannabinoids used in the experiments were either synthetic cannabinoids obtained from Restek Corporation USA, or plant derived purified cannabinoids. Cannabinoid solutions were prepared by dissolving the cannabinoid in dimthylsulphoxide (DMSO) at the desired concentration.

Cell lines—cell lines used in the experiments were obtained from ATCC®. Human colon cancer cell lines that were used in the experiments were: HCT-116, RKO, HT-29, HT-8, LS-174T, SK-CO1. Mouse colon cancer cell line: CT-26.

Lung cancer cell lines used in the experiments were: A549, DMS-79, NCI-H69, NCI-H211, and SHP77.

Breast cancer cell lines were: AU565, BT-549, MCF-7, and SW-527. All cell lines were incubated with mediums according to the manufacturer's instructions.

### Tests

Cell viability was performed by CellTiter-Glo (CTG) Luminescent Cell Viability Assay. IC₅₀ of each cannabinoid on the cancer cell lines was determined by standard methods and the use of the software GraphPad Prism.

Synergistic calculation—synergism was calculated according to the Bliss equation defined supra.

### Example 1—Certain Cannabinoid Combinations Exhibit Colon, Breast, and Lung Cancers Cells Viability Inhibition Effect

Several colon cancer cell lines, i.e., HT-29, HCT-8, HCT-116, LS-174T, CT-26, RKO, and SK-CO1; lung cancer cell lines, i.e., A549, DMS-79, NCI-H69, NCI-H211, and SHP77; and breast cancer cell lines, i.e., AU565, BT-549, MCF-7, and SW-527 were incubated at 37° C. with various cannabinoids for 48 hours. As can be seen in Tables 1-3 below, various combinations of the cannabinoids: CBCA, CBDV, CBDVA, THCV, and CBGA, at concentrations range of 11.5 μM-51.2 μM, illustrated a synergistic proliferation/viability inhibition effect on the cancer cells. As can be further seen, the combinations of CBCA and THC-d8, and of CBGA and THC-d8 yielded no synergistic anti-cancer effect on colon cancer cells. Further, CBCA and CBL, and CBDVA and THC-d8 did not exhibit a synergistic effect on lung and breast cancer cells, suggesting that only certain cannabinoid combinations possess a synergistic anti-cancer effect.

### Example 2—the Combination of CBDV and CBGA Possesses a Strong Synergistic Anti-Proliferative Effect on HT-29 Colon Cancer Cells

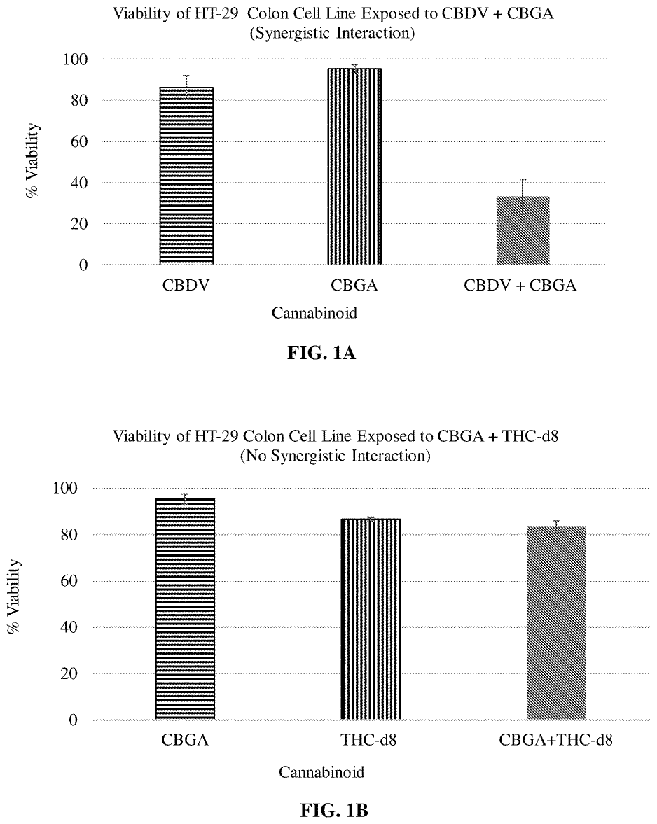

The colon cancer cell line HT-29 was exposed to each of CBDV (at a concentration of 23.5 μM) and CBGA (at a concentration of 25.6 μM) alone, and a combination thereof and to each of CBGA (at a concentration of 25.6 μM) and THC-d8 (at a concentration of 17.5 μM) alone, and a combination thereof at 37° C. for 48 hours. The viability of the tested cells was examined. The following Tables 4 and 5 illustrate the obtained results (shown also graphically at FIGS. 1A and 1B). As can be seen in table 4 and in FIG. 1A, each of CBDV and CBGA alone illustrated a slight growth inhibition effect. Surprisingly, the combination of the two cannabinoids presents a substantial and strong synergistic anti-cancer effect. Conversely, as can be seen in table 5 and in FIG. 1B, the combination of CBGA with THC-d8 illustrated no anti-cancer synergistic effect, suggesting that CBGA possesses synergistic anti-cancer effect when combined with certain cannabinoids only.

TABLE 4
the combination of CBDV and CBGA possesses a strong
synergistic growth inhibition effect on HT-29 colon cancer cells.
CBDV + CBGA

| Cannabinoid | Viability | SD |
|---|---|---|
| CBDV | 86 | 6 |
| CBGA | 95 | 2 |
| CBDV + CBGA | 33 | 8 |
| Synergistic effect | | 0.491253 |

X > 0 = Synergy,
X < 0 = Antagonism

TABLE 5
the combination of CBGA and THC-d8 does not possess a
synergistic growth inhibition effect on HT-29 colon cancer cells.
CBGA + THC-d8

| Cannabinoid | Viability | SD |
|---|---|---|
| CBGA | 95 | 2 |
| THC-d8 | 87 | 1 |
| CBGA + THC-d8 | 83 | 3 |
| Synergistic effect | | −0.0092 |

X > 0 = Synergy,
X < 0 = Antagonism

### Example 3—the Combination of CBDV and CBGA Possesses a Strong Synergistic Anti-Proliferative Effect on A549 Lung Cancer Cells

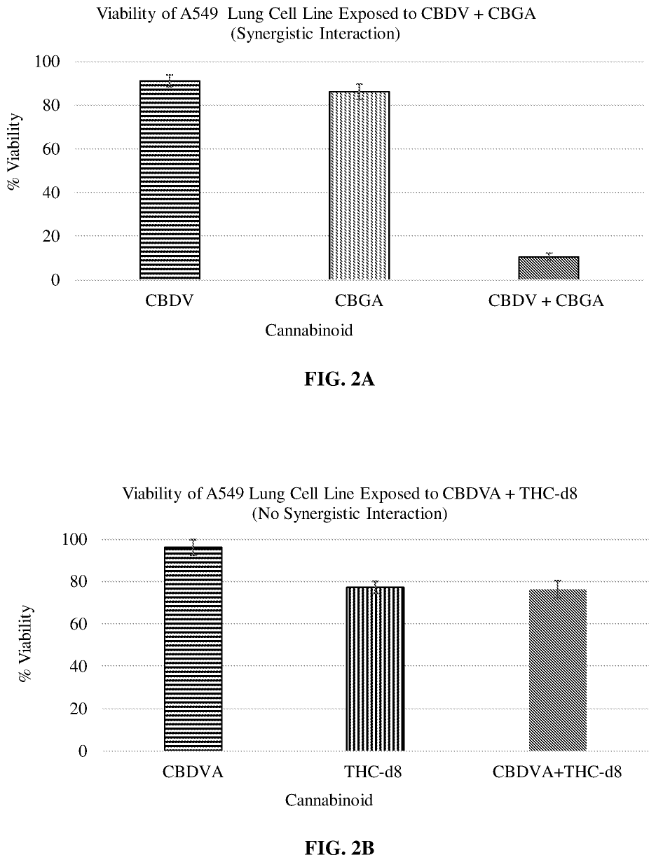

The lung cancer cell line A549 was exposed to each of CBDV (at a concentration of 23.5 μM) and CBGA (at a concentration of 25.6 μM) alone, and a combination thereof at 37° C. for 48 hours. The viability of the tested cells was examined. The following Table 6 illustrates the obtained results (shown also graphically in FIGS. 2A-2B). As can be seen in table 6 and in FIG. 2A, at the tested concentrations, each of CBDV and CBGA alone illustrated a slight growth inhibition effect. Surprisingly, the combination of the two cannabinoids presents a substantial and strong synergistic anti-cancer effect. As can be seen in table 7 and in FIG. 2B, the combination of CBDVA (at a concentration of 25.6 μM) with THC-d8 (at a concentration of 17.5 μM), illustrated no anti-cancer synergistic effect on the lung cancer cells suggesting that only certain cannabinoids' combinations possess synergistic anti-cancer effect.

TABLE 6
the combination of CBDV and CBGA possesses a strong
synergistic growth inhibition effect on A549 lung cancer cells.
CBDV + CBGA

| Cannabinoid | Viability | SD |
|---|---|---|
| CBDV | 91 | 3 |
| CBGA | 86 | 3 |
| CBDV + CBGA | 11 | 2 |
| Synergistic effect | | 0.679188 |

X > 0 = Synergy,
X < 0 = Antagonism

TABLE 7
CBDVA and THC-d8 do not possess a synergistic
growth inhibition effect on A549 lung cancer cells.
CBDVA + THC-d8

| Cannabinoid | Viability | SD |
|---|---|---|
| CBDVA | 96 | 4 |
| THC-d8 | 77 | 3 |
| CBDVA + THC-d8 | 76 | 4 |
| Synergistic effect | | −0.01989 |

X > 0 = Synergy,
X < 0 = Antagonism

### Example 4—the Combination of CBDV and CBGA Possesses a Strong Synergistic Anti-Proliferative Effect on MCF-7 Breast Cancer Cells

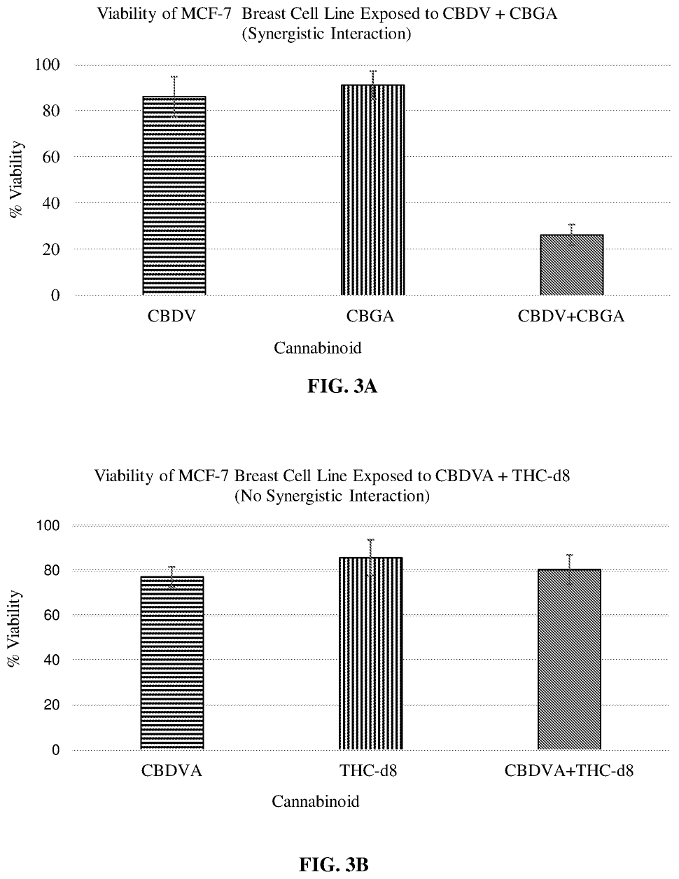

The breast cancer cell line MCF-7 was exposed to each of CBDV (at a concentration of 23.5 μM) and CBGA (at a concentration of 20 μM) alone, and to a combination thereof at 37° C. for 48 hours. The viability of the tested cells was examined. The following Table 8 illustrates the obtained results (shown also graphically in FIGS. 3A-3B). As can be seen in table 8 and in FIG. 3A, each of CBDV and CBGA alone illustrated a slight growth inhibition effect. Surprisingly, the combination of the two cannabinoids presents a substantial and strong synergistic anti-cancer effect. As can be seen in table 9 and in FIG. 3B, the combination of CBDVA (at a concentration of 17.5 μM) with THC-d8 (at a concentration of 17.5 μM), illustrated no anti-cancer synergistic effect suggesting that only certain cannabinoids' combinations, possess synergistic anti-cancer effect.

TABLE 8
CBDV and CBGA possess a strong synergistic growth
inhibition effect on MCF-7 breast cancer cells.
CBDV + CBGA

| Cannabinoid | Viability | SD |
|---|---|---|
| CBDV | 86 | 9 |
| CBGA | 91 | 6 |
| CBDV + CBGA | 26 | 4 |
| Synergistic effect | | 0.521795 |

X > 0 = Synergy,
X < 0 = Antagonism

TABLE 9
CBDVA and THC-d8 do not possess a synergistic growth
inhibition effect on MCF-7 breast cancer cells.
CBDVA + THC-d8

| Cannabinoid | Viability | SD |
|---|---|---|
| CBDVA | 77 | 4 |
| THC-d8 | 86 | 8 |
| CBDVA + THC-d8 | 80 | 7 |
| Synergistic effect | | −0.1428 |

X > 0 = Synergy,
X < 0 = Antagonism

### Example 5—the Combination of CBDV and CBGA Possesses a Synergistic Anti-Proliferative Effect on RKO and HCT116 Colon Cancer Cells

The colon cancer cell lines RKO, and HCT116 were exposed to various cannabinoid combinations, at 37° C. for 48 h. The viability of the tested cells was examined. Further, synergism was evaluated using the Bliss equation. Table 10 below illustrates the obtained results.

TABLE 10
synergic effect of two cannabinoids
on human colon cancer cell lines

| | |
|---|---|
| HCT-116 | RKO |

| | | Bliss | | | Bliss |
|---|---|---|---|---|---|
| A | B | value | A | B | value |
| CBDV | CBCA | 0.34 | CBDV | CBCA | 0.29 |
| CBDV | CBGA | 0.41 | CBDV | CBGA | 0.31 |
| CBDV | CBDVA | 0.16 | CBDV | CBDVA | 0.19 |
| THCV | CBCA | 0.45 | THCV | CBCA | 0.16 |
| THCV | CBDVA | 0.39 | THCV | CBDVA | 0.14 |
| THCV | CBGA | 0.27 | THCV | CBGA | 0.12 |

The combined effect of CBDV and CBGA presented a particularly strong synergistic interaction and were therefore further tested. The colon cancer cell lines RKO, and HCT116 were exposed to CBDV and CBGA combinations, at 37° C. for 48 h. The viability of the tested cells was examined.

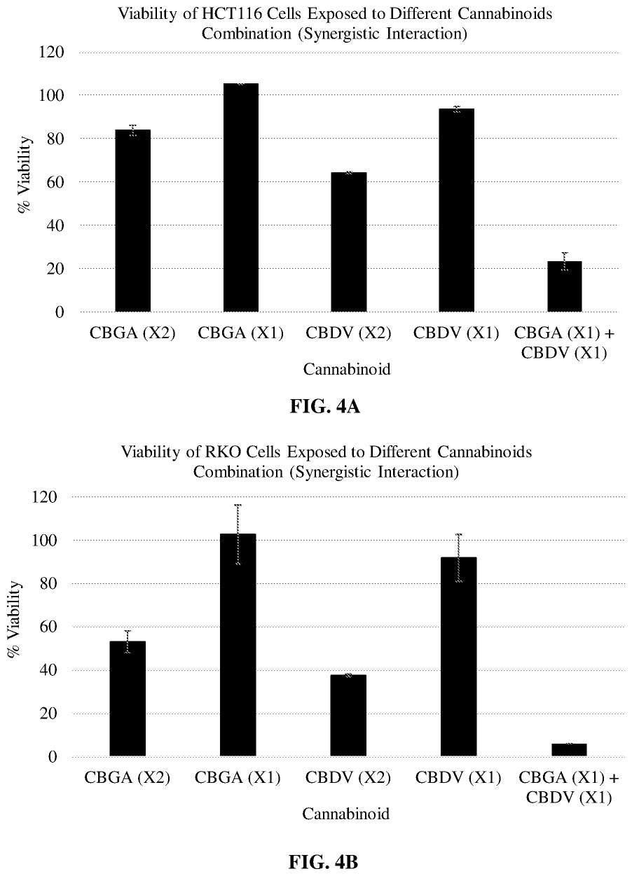

CBDV and CBGA were tested at concentrations of 46.9 μM and 51.2 μM, respectively (shown as X2 in FIGS. 4A-4B). The effect of contacting each cannabinoid on the cells at half the concentration (i.e., 23.4 μM and 25.6 μM for CBDV and CBGA, respectively) was further tested (shown as X1 in FIGS. 4A-4B). Mixtures of CBDV and CBGA each at a X1 concentration were further prepared (“low concentration solutions”). The effect of the low concentration solution on the viability of the cancer cells was tested. The testing of the effect of each solution on the cancer cells was determined by incubating the cells with each cannabinoid solution as well as a standard (control) comprising DMSO only at 37° C. for 48 h and cell viability was measured by CTG viability assay using a Varioskan™ LUX multimode microplate reader. The test results are presented in Tables 11-12 and in FIGS. 4A-4B.

The absolute cell viability results upon exposure of HCT-116 human colon cancer cell lines to (i) CBDV, CBGA at concentration X2, (ii) CBDV, CBGA at X1 and (iii) to the binary mixture CBDV and CBGA each at the X1 concentration are presented in Table 11.

The absolute cell viability results upon exposure of RKO human colon cancer cell lines to (i) CBDV, CBGA at concentration X2, (ii) CBDV, CBGA at X1 and (iii) to the binary mixture CBDV and CBGA each at the X1 concentration are presented in Table 12.

As can be seen in FIGS. 4A-4B, and in tables 11 and 12, there was a substantial decrease in RKO and HCT116 cells' viability after exposure to binary cannabinoids mixture (CBGA+CBDV) relative to the largest additive effect on the cells when exposed to a single cannabinoid at X1. The results show that all binary cannabinoid mixtures exhibit better anti-proliferation effect than the calculated additive effect or the double concentration of a single cannabinoid on both HCT-116 and RKO human colon cancer cell lines. The CBDV+CBDG binary mixture exhibits the highest effect on both cell lines.

Based on these results, the expected calculated (SAB) (Cal) viability value of the CBDV and CBGA binary composition according to the Bliss independence drug interaction model is 98.5 and 94.3 for HCT 116 and RKO, respectively. The actual values that were obtained in the experiments were (SAB) (Exp) of 23.4 and 6, suggest that the CBDV and CBGA binary mixture exhibits a synergistic effect (see below in tables 11 and 12).

TABLE 11
CBDV and CBGA possess a strong synergistic
growth inhibition effect on HCT116 cancer cells.
CBGA + CBDV (HCT116)

| Cannabinoid | Viability | SE |
|---|---|---|
| CBGA (X2) | 84 | 2 |
| CBGA (X1) | 105 | 0 |
| CBDV (X2) | 64 | 0 |
| CBDV (X1) | 94 | 1 |
| CBGA (X1) + CBDV (X1) | 23 | 4 |
| Synergistic effect | | 0.7511 |

X > 0 = Synergy,
X < 0 = Antagonism

TABLE 12
CBDV and CBGA possess a strong synergistic
growth inhibition effect on RKO cancer cells.
CBGA + CBDV (RKO)

| Cannabinoid | Viability | SE |
|---|---|---|
| CBGA (X2) | 53 | 5 |
| CBGA (X1) | 103 | 14 |
| CBDV (X2) | 38 | 1 |
| CBDV (X1) | 92 | 11 |
| CBGA (X1) + CBDV (X1) | 6 | 0 |
| Synergistic effect | | 0.8828 |

X > 0 = Synergy,
X < 0 = Antagonism

### Example 6—Synergy Ratio Between CBDV and CBGA

Human colon cell lines (SC-CO1, RKO, and HCT116) and a murine cell line (CT26) were incubated with varying molar ratios between CBGA and CBDV of 1:3.5 to 8.5:1. Cells' viability and corresponding synergy rankings were determined. As can be seen in tables 13-16, most ratios tested showed synergistic interaction between CBDV and CBGA.

TABLE 13A
% cells viability of HCT 116 cells
% Cells Viability

| | | | | | | | | |
|---|---|---|---|---|---|---|---|---|
| CBDV | DMSO | 72 | 90 | 97 | 104 | 112 | 115 | 100 |
| | 5 uM | 37 | 60 | 63 | 79 | 99 | 104 | 113 |
| | 10 uM | 22 | 11 | 46 | 60 | 80 | 89 | 109 |
| | 15 uM | 8 | 24 | 33 | 42 | 56 | 72 | 101 |
| | 25 uM | 4 | 8 | 15 | 24 | 38 | 48 | 84 |
| | 30 uM | 1 | 1 | 2 | 5 | 27 | 38 | 71 |
| | 35 uM | 0 | 1 | 1 | 3 | 12 | 19 | 64 |
| | | 42.5 uM | 35 uM | 30 uM | 25 uM | 15 uM | 10 uM | DMSO |

CBGA

TABLE 13B
HCT 116 cells -CBDV and CBGA ratio
Cannabinoid Ratio

| | | | | | | | |
|---|---|---|---|---|---|---|---|
| CBDV | 5 uM | 1:8.5 | 1:7 | 1:6 | 1:5 | 1:3 | 1:2 |
| | 10 uM | 1:4.3 | 1:3.5 | 1:3 | 1:2.5 | 1:1.5 | 1:1 |
| | 15 uM | 1:2.8 | 1:2.3 | 1:2 | 1:1.6 | 1:1 | 1:1.5 |
| | 25 uM | 1:1.7 | 1:1.4 | 1:1.2 | 1:1 | 1:1.6 | 1:2.5 |
| | 30 uM | 1:1.4 | 1:1.2 | 1:1 | 1:1.2 | 1:2 | 1:3 |
| | 35 uM | 1:1.2 | 1:1 | 1:1.2 | 1:1.4 | 1:2.3 | 1:3.5 |
| | | 42.5 uM | 35 uM | 30 uM | 25 uM | 15 uM | 10 uM |

CBGA

TABLE 13C
HCT 116 cells -synergy according to Bliss equation
Synergy

| | | | | | | | |
|---|---|---|---|---|---|---|---|
| CBDV | 5 uM | 0.44 | 0.42 | 0.46 | 0.38 | 0.27 | 0.25 |
| | 10 uM | 0.57 | 0.87 | 0.59 | 0.53 | 0.42 | 0.36 |
| | 15 uM | 0.65 | 0.67 | 0.65 | 0.64 | 0.58 | 0.45 |
| | 25 uM | 0.56 | 0.67 | 0.66 | 0.64 | 0.56 | 0.48 |
| | 30 uM | 0.50 | 0.63 | 0.67 | 0.70 | 0.53 | 0.44 |
| | 35 uM | 0.46 | 0.57 | 0.60 | 0.63 | 0.59 | 0.54 |
| | | 42.5 uM | 35 uM | 30 uM | 25 uM | 15 uM | 10 uM |

CBGA

TABLE 14A
% cells viability of RKO cells
% Cells Viability

| | | | | | | | | |
|---|---|---|---|---|---|---|---|---|
| CBDV | DMSO | 41 | 59 | 75 | 94 | 119 | 119 | 101 |
| | 5 uM | 9 | 22 | 26 | 46 | 71 | 89 | 112 |
| | 10 uM | 4 | 1 | 11 | 28 | 51 | 67 | 108 |
| | 15 uM | 2 | 6 | 7 | 16 | 29 | 51 | 88 |
| | 25 uM | 1 | 2 | 2 | 6 | 10 | 28 | 62 |
| | 30 uM | 1 | 1 | 1 | 1 | 8 | 15 | 53 |
| | 35 uM | 1 | 1 | 1 | 1 | 3 | 8 | 48 |
| | | 42.5 uM | 35 uM | 30 uM | 25 uM | 15 uM | 10 uM | DMSO |

CBGA

TABLE 14B
RKO cells -CBDV and CBGA ratio
Cannabinoid Ratio

| | | | | | | | |
|---|---|---|---|---|---|---|---|
| GBDV | 5 uM | 1:8.5 | 1:7 | 1:6 | 1:5 | 1:3 | 1:2 |
| | 10 uM | 1:4.3 | 1:3.5 | 1:3 | 1:2.5 | 1:1.5 | 1:1 |
| | 15 uM | 1:2.8 | 1:2.3 | 1:2 | 1:1.6 | 1:1 | 1:1.5 |
| | 25 uM | 1:1.7 | 1:1.4 | 1:1.2 | 1:1 | 1:1.6 | 1:2.5 |
| | 30 uM | 1:1.4 | 1:1.2 | 1:1 | 1:1.2 | 1:2 | 1:3 |
| | 35 uM | 1:1.2 | 1:1 | 1:1.2 | 1:1.4 | 1:2.3 | 1:3.5 |
| | | 42.5 uM | 35 uM | 30 uM | 25 uM | 15 uM | 10 uM |

CBGA

TABLE 14C
RKO cells -synergy according to Bliss equation
Synergy

| | | | | | | | |
|---|---|---|---|---|---|---|---|
| CBDV | 5 uM | 0.37 | 0.43 | 0.59 | 0.60 | 0.63 | 0.44 |
| | 10 uM | 0.40 | 0.62 | 0.70 | 0.73 | 0.78 | 0.61 |
| | 15 uM | 0.33 | 0.46 | 0.59 | 0.66 | 0.76 | 0.54 |
| | 25 uM | 0.24 | 0.35 | 0.45 | 0.53 | 0.64 | 0.46 |
| | 30 uM | 0.21 | 0.31 | 0.39 | 0.49 | 0.56 | 0.48 |
| | 35 uM | 0.19 | 0.27 | 0.35 | 0.44 | 0.54 | 0.49 |
| | | 42.5 uM | 35 uM | 30 uM | 25 uM | 15 uM | 10 uM |

CBGA

TABLE 15A
% cells viability of SK-CO1 cells
% Cells Viability

| | | | | | | | | |
|---|---|---|---|---|---|---|---|---|
| CBDV | DMSO | 52 | 76 | 91 | 101 | 110 | 108 | 101 |
| | 5 uM | 46 | 65 | 76 | 96 | 104 | 106 | 104 |
| | 10 uM | 33 | 11 | 60 | 87 | 100 | 104 | 103 |
| | 15 uM | 18 | 39 | 53 | 73 | 92 | 99 | 99 |
| | 25 uM | 19 | 25 | 42 | 51 | 80 | 90 | 99 |
| | 30 uM | 7 | 10 | 24 | 38 | 73 | 80 | 97 |
| | 35 uM | 1 | 7 | 24 | 26 | 58 | 76 | 96 |
| | | 42.5 uM | 35 uM | 30 uM | 25 uM | 15 uM | 10 uM | DMSO |

CBGA

TABLE 15B
SK-CO1 cells -CBDV and CBGA ratio

| | | | | | | | |
|---|---|---|---|---|---|---|---|
| CBDV | 5 uM | 1:8.5 | 1:7 | 1:6 | 1:5 | 1:3 | 1:2 |
| | 10 uM | 1:4.3 | 1:3.5 | 1:3 | 1:2.5 | 1:1.5 | 1:1 |
| | 15 uM | 1:2.8 | 1:2.3 | 1:2 | 1:1.6 | 1:1 | 1:1.5 |
| | 25 uM | 1:1.7 | 1:1.4 | 1:1.2 | 1:1 | 1:1.6 | 1:2.5 |
| | 30 uM | 1:1.4 | 1:1.2 | 1:1 | 1:1.2 | 1:2 | 1:3 |
| | 35 uM | 1:1.2 | 1:1 | 1:1.2 | 1:1.4 | 1:2.3 | 1:3.5 |
| | | 42.5 uM | 35 uM | 30 uM | 25 uM | 15 uM | 10 uM |

CBGA

TABLE 15C
SK-CO1 cells -synergy according to Bliss equation
Synergy

| | | | | | | | |
|---|---|---|---|---|---|---|---|
| CBDV | 5 uM | 0.08 | 0.15 | 0.19 | 0.10 | 0.11 | 0.07 |
| | 10 uM | 0.21 | 0.67 | 0.34 | 0.17 | 0.14 | 0.08 |
| | 15 uM | 0.34 | 0.36 | 0.37 | 0.28 | 0.17 | 0.09 |
| | 25 uM | 0.33 | 0.51 | 0.49 | 0.49 | 0.29 | 0.18 |
| | 30 uM | 0.43 | 0.64 | 0.63 | 0.59 | 0.33 | 0.25 |
| | 35 uM | 0.49 | 0.66 | 0.63 | 0.70 | 0.47 | 0.28 |
| | | 42.5 uM | 35 uM | 30 uM | 25 uM | 15 uM | 10 uM |

CBGA

TABLE 16A
% cells viability of CT26 cells
% Cells Viability

| | | | | | | | | |
|---|---|---|---|---|---|---|---|---|
| CBDV | DMSO | 11 | 21 | 22 | 46 | 102 | 102 | 100 |
| | 5 uM | 11 | 28 | 27 | 60 | 107 | 112 | 108 |
| | 10 uM | 5 | 2 | 25 | 59 | 94 | 110 | 109 |
| | 15 uM | 3 | 5 | 12 | 44 | 76 | 91 | 113 |
| | 25 uM | 1 | 3 | 6 | 21 | 53 | 78 | 108 |
| | 30 uM | 2 | 1 | 4 | 6 | 35 | 61 | 99 |
| | 35 uM | 0 | 3 | 3 | 3 | 18 | 28 | 90 |
| | | 42.5 uM | 35 uM | 30 uM | 25 uM | 15 uM | 10 uM | DMSO |

CBGA

TABLE 16B
CT26 cells -CBDV and CBGA ratio

| | | | | | | | |
|---|---|---|---|---|---|---|---|
| CBDV | 5 uM | 1:8.5 | 1:7 | 1:6 | 1:5 | 1:3 | 1:2 |
| | 10 uM | 1:4.3 | 1:3.5 | 1:3 | 1:2.5 | 1:1.5 | 1:1 |
| | 15 uM | 1:2.8 | 1:2.3 | 1:2 | 1:1.6 | 1:1 | 1:1.5 |
| | 25 uM | 1:1.7 | 1:1.4 | 1:1.2 | 1:1 | 1:1.6 | 1:2.5 |
| | 30 uM | 1:1.4 | 1:1.2 | 1:1 | 1:1.2 | 1:2 | 1:3 |
| | 35 uM | 1:1.2 | 1:1 | 1:1.2 | 1:1.4 | 1:2.3 | 1:3.5 |
| | | 42.5 uM | 35 uM | 30 uM | 25 uM | 15 uM | 10 uM |

CBGA

TABLE 16C
CT26 cells -synergy according to Bliss equation
Synergy

| | | | | | | | |
|---|---|---|---|---|---|---|---|
| CBDV | 5 uM | 0.01 | −0.05 | −0.03 | −0.10 | 0.03 | −0.02 |
| | 10 uM | 0.07 | 0.21 | −0.01 | −0.09 | 0.18 | 0.02 |
| | 15 uM | 0.09 | 0.18 | 0.13 | 0.08 | 0.39 | 0.24 |
| | 25 uM | 0.10 | 0.19 | 0.18 | 0.28 | 0.57 | 0.32 |
| | 30 uM | 0.09 | 0.20 | 0.18 | 0.39 | 0.67 | 0.41 |
| | 35 uM | 0.09 | 0.16 | 0.17 | 0.38 | 0.73 | 0.64 |
| | | 42.5 uM | 35 uM | 30 uM | 25 uM | 15 uM | 10 uM |

CBGA

### Example 7—the Combinations of CBCA, CBDV, CBDVA, THCV and CBGA Produce a Synergistic Cell Viability Inhibitory Effect on Colon Cancer Biopsies

The anti-tumor effect of combinations of cannabinoids CBCA, CBDV, CBDVA, THCV and CBGA on human colon cancer biopsies was tested. As can be seen in table 17 below, CBCA, CBDV, CBDVA, THCV and CBGA combinations significantly inhibit the cells' viability of human colon biopsies. The obtained cell viability inhibition produced synergistic effects.

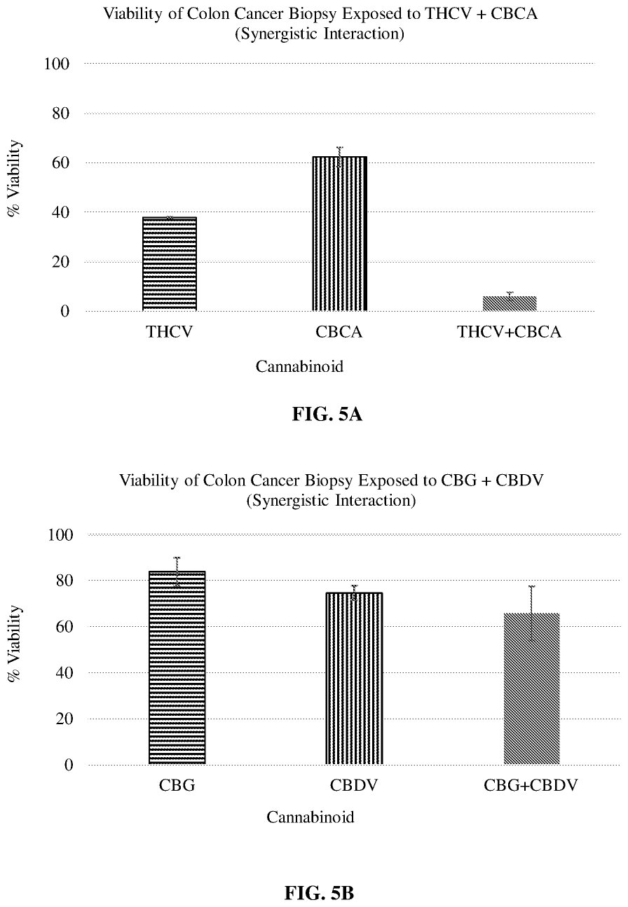

Table 18 illustrates (shown also graphically at FIG. 5A) the synergistic anti-tumor effect of a combination of the cannabinoids THCV (at a concentration of 24 μM) and CBCA (at a concentration of 14.5 μM) on colon cancer biopsies. As can be seen, the combination of THCV and CBCA yielded a synergistic cell viability inhibitory effect. As can be seen in table 19 (shown also graphically in FIG. 5B), the combination of CBDV (at a concentration of 23.5 μM) with CBG (at a concentration of 20 μM) illustrated no anti-cancer synergistic effect suggesting that only certain cannabinoid combinations possess synergistic anti-cancer effect.

TABLE 18
THCV and CBCA possess a strong synergistic
growth inhibition effect on colon cancer biopsy.
THCV + CBCA

| Cannabinoid | Viability | SD |
|---|---|---|
| THCV | 38 | 0.3 |
| CBCA | 62 | 4 |
| THCV + CBCA | 6 | 2 |
| Synergistic effect | | 0.1769 |

X > 0 = Synergy,
X < 0 = Antagonism

TABLE 19
CBG and CBDV possess no synergistic effect on colon cancer biopsy.
CBG + CBDV

| Cannabinoid | Viability | SD |
|---|---|---|
| CBG | 84 | 6 |
| CBDV | 75 | 3 |
| CBG + CBDV | 66 | 12 |
| Synergistic effect | | −0.03124 |

X > 0 = Synergy,
X < 0 = Antagonism

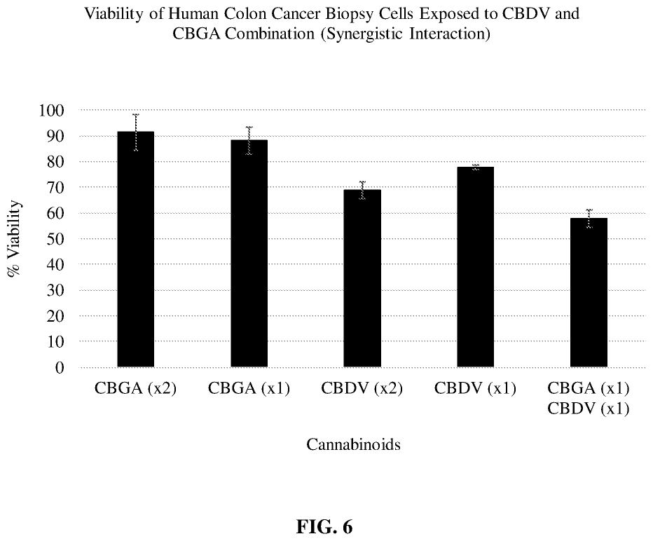

FIG. 6 illustrates graphically the combination of CBGA and CBDV on colon cancer biopsies taken from a patient. The concentration of each component was: CBGA (X2-51.2 μM), CBGA (X1-25.6 μM), CBDV (X2-47 μM), CBGA (X1-23.5 μM). As can be seen in FIG. 6, the combination of CBGA(X1) and CBDV(X1) yielded a synergistic cell viability inhibitory effect. Surprisingly, the effect of the combination of the cannabinoids on the cell viability was more profound than each of the cannabinoids alone at the higher (X2 concentration).

### Example 8—Synergistic Anti-Tumor Effect of a Combination of the Cannabinoids CBDV and CBGA and Chemotherapy on Colon Cancer Cells

Human colon cell lines (HT-29, HCT8, HCT116, LS-174T, RKO and CT-26) were incubated with various chemotherapeutic agents to test the effect of the combined cannabinoids CBDV and CBGA on chemotherapy treatment. As can be seen in table 20, certain cannabinoid combinations with Docetaxel, Epirubicine, 5-Fluorouracil, Capecitabine, and Oxaliplatin demonstrated a strong synergistic anti-cancer effect on most colon cancer cells tested.

FIGS. 7A-7B illustrate the human colon cell lines (HCT116 and RKO) that were incubated with CBGA at a concentration of 35 μM, CBDV at a concentration of 25 μM, Capecitabine at a concentration of 250 μM, and a combination thereof. As can be seen in FIGS. 7A and 7B, capecitabine alone had no toxicity effect on the cells. The combination of CBGA and CBDV had a substantial anti-cancer effect on the cells. The combination of the three agents together, i.e., CBGA, CBDV and Capecitabine had a significant anti-cancer effect which was higher than the effect of the combined treatment of CBGA and CBDV, resulting in a growth inhibition of above about 80% of the cells. Taken together, these results demonstrate a synergistic anti-proliferative effect of CBDV and CBGA and the chemotherapy tested.

As can be seen in FIG. 8A, docetaxel (at a concentration of 80 μM) also presented synergistic interaction with the combination of CBDV (at a concentration of 10 μM) and CBGA (at a concentration of 10 μM).

As can be seen in FIG. 8B, although the combined anti-cancer effect of CBDV (at a concentration of 35 μM) and CBGA (at a concentration of 35 μM) was reduced in the presence of Irinotecan (at a concentration of 80 μM), the combination with those two cannabinoids did not hinder the effect of Irinotecan.

Taken together, the above results suggest that the herein cannabinoids can be administered along with the treatment of chemotherapies agents.

### Example 9—the Combination of CBGA and CBDV Substantially Inhibits Growth of Colon Cancer Tumors in Mice

Nude mice (Athymic nude mice, about 6-8 weeks of age), 7 mice per group, were inoculated subcutaneously with HT-29 human colorectal adenocarcinoma cells. After reaching a tumor volume of 100 mm³, the mice were intraperitoneally injected daily (for 16 consecutive days) with 200 μl of a combination of CBDV and CBGA or control. Tumor volume was measured 3 times per week. The experiment concluded when the first mouse (control group) reached the maximal tumor volume allowed, 16 days after xenograft inoculation.

Mice acclimation: At least 5 days

Induction of Model: HT-29: By a single subcutaneous (SC) injection of 1×10⁶ cells/animal mixed with Matrigel (1:1 v/v) at a dose volume of 200 μl/Animal on day 0.

Randomization: Randomization of mice to test groups was carried out when tumors reach a mean volume of 100 mm³ (tumor size range of 70-120 mm³) and up to 2 weeks from injection of tumor cells.

Monitoring of tumor size: was performed by measurement of tumor size up to 3× weekly.

Treatment (vehicle or cannabinoid combination): once daily repeated IP injections for 4 successive weeks at a volume of 200 μl.

Examinations (Morbidity & Mortality)—Once daily

Detailed Clinical Signs—Once weekly

Body weight—Twice weekly

Tumor Measurement—3× weekly. Tumor diameters were measured with digital calipers and the tumor volume (mm³) was be calculated by the formula:

Volume=(width)2×length/2

Study Duration: Up to 6 weeks (1-2 weeks for tumor development & 4-week treatment period). Study may be extended in due course of the study according to the results obtained.

Terminal Procedures: when mice were euthanized, the tumor was be excised, weighed, and photographed next to a ruler.

Formulation Preparation:

The experiment formulation contains:
Two cannabinoids at final concentration of 1 mg from each in volume of 200 μl for injection CBDV at a concentration of 17.46 mM and CBGA at a concentration of 13.87 mM; 10% w/v for each cannabinoid). Injection solution at ratio of 1:1:1:17 respectively contained: Absolute Ethanol, Dimethyl sulfoxide, Kolliphore, and Saline.

Procedure:

118 mg from each Cannabinoid was weighed separately and then combined in a 50 mL tube. Dissolving solution was prepared in ratio of 1:1:1 for Ethanol, Dimethyl sulfoxide and Kolliphore. Prior to injection a saline solution was added and 200 μL was finally injected into each mouse daily.

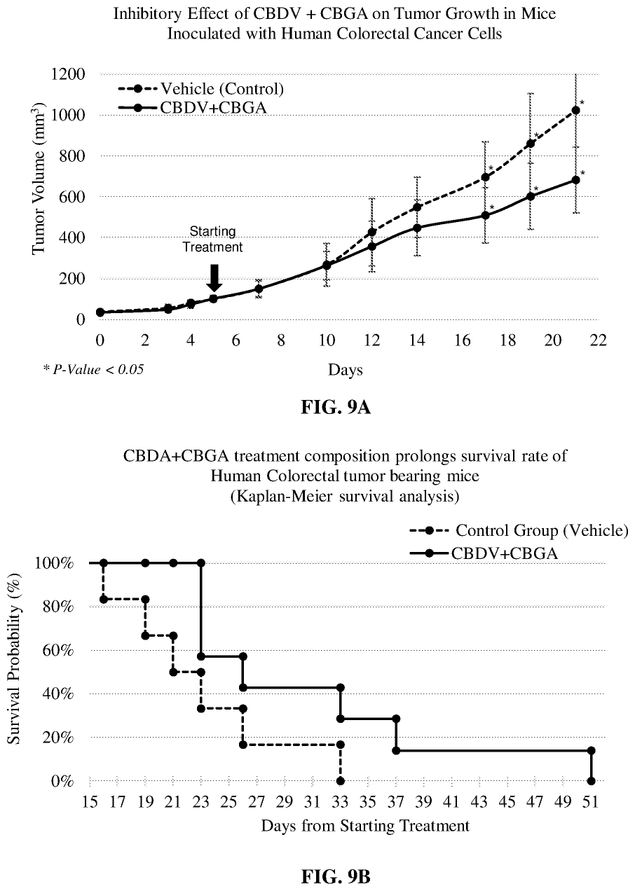

As can be seen in FIG. 9A, significant reduction in tumor volume of 33% (p value=0.016) was observed in the CBDV and CBGA treated mice. Thus, the herein results indicate that CBGA and CBDV are effective in treating colon cancer. As can be seen in FIG. 9B, mice treated with CBDV and CBGA combination illustrated prolonged survival as compared to control mice.

### Example 10—Clinical Studies for Determining the Effect of CBDV, CBGA and THC on Colon Cancer Patients

The efficacy of compound CN-101 (CBDV 100 mg, CBGA 100 mg, THC 5 mg) as treatment for colon cancer can be done by a randomized, parallel-group, double-blind, multicenter clinical study as set forth below.

Condition or disease: Colon Cancer

Intervention/treatment: Drug: Conventional chemotherapy; Drug: CN-101; Drug: Placebo CN-101

Detailed Description:

Although many therapies exist and are being developed to relieve symptoms for colon cancer, there are few randomized controlled clinical trials to evaluate cannabinoid-based interventions in colon cancer. The aim of this study is to evaluate the anti-tumor effect of compound CN-101 based on chemotherapy for colon cancer patients. This will be multi-center, randomized, double-blind, placebo-controlled study to evaluate the synergistic anti-tumor effect of CN-101 on colon cancer patients. Following a run-in period, approximately 60 subjects will be randomly assigned to conventional chemotherapy treatment group, CN-101 and conventional chemotherapy treatment group for 26 weeks. After the 26 weeks treatment period, subjects in two treatments arms will follow-up 36 months.

Study Design

### Study Type: Interventional (Clinical Trial)

Estimated Enrollment: 60 participants

### Allocation: Randomized

### Intervention Model: Parallel Assignment

### Masking: Triple (Participant, Investigator, Outcomes Assessor)

### Primary Purpose: Treatment

Official Title: Anti-tumor Effect of cn-101 Based on Chemotherapy for Colon Cancer

### Patients: a Randomized, Parallel-group, Double-blind, Multicenter Clinical Study

Outcome Measures

Primary Outcome Measures:

Number of Participants With Adverse Event (AE) [Time Frame: The time frame for adverse event reporting is from the first dose date to 30 days since the last dose date]. Severity is graded using Common Terminology Criteria for Adverse Events (CTCAE) v3.0, with the exception of some dermatology/skin adverse events that are graded using CTCAE v3.0 with modifications. Fatal adverse events are classified as grade 5. Serious adverse events include any event that is fatal, life threatening, requires inpatient hospitalization or prolongation of existing hospitalization, results in persistent or significant disability/incapacity, a congenital anomaly/birth defect, or other significant medical hazard. Treatment-related AEs are those that the investigator will consider a reasonable possibility that might be caused by study drug.

Quality of life [Time Frame: Change from Baseline in the QLQ-C30 (V3.0) at the week 2, 4, 6, 8, 10, 13, 26 of the treatment phases, the week 13, 26, 39 and 52, 65, 78, 91, 104, 117, 130 of the follow-up phases]

Using QLQ-C30 (V3.0) to assess the impact of COPD on a person's life, and how this change over time.

Secondary Outcome Measures:

Symptom [Time Frame: Change from Baseline in symptom score at the week 2, 4, 6, 8, 10,13, 26 of the treatment phases, the week 13, 26, 39 and 52, 65, 78, 91, 104, 117, 130 of the follow-up phases]

Investigator-assessed Progression-Free Survival (PFS) [Time Frame: 3 years] The time from the date of randomization until objective tumor progression or death due to any cause. Objective tumor progression is determined through radiological imaging and based on the requirements of the Response Evaluation Criteria in Solid Tumors (RECIST Version 1.1)

Progression-free Survival (PFS) [Time Frame: the time from the date of randomization to the date of first disease progression, or death within 60 days after the last evaluable tumor assessment or randomization date (whichever was later)]

Eligibility Criteria

### Ages Eligible for Study: 18 Years to 75 Years (Adult, Older Adult)

### Sexes Eligible for Study: All

### Accepts Healthy Volunteers: No

### Inclusion Criteria:

### Exclusion Criteria:

patients with other primary malignant tumors within 1 year.
intentional, severe liver and kidney disease patients with serious obstacle and function.
pregnancy or lactating women, mental disorders to cooperate to complete the study.
is in other subjects or attended other drugs test interval <3 months.
   do not meet the inclusion criteria.

1-27. (canceled)

28. A pharmaceutical cannabinoid composition for treating cancer, the composition comprising at least two cannabinoids selected from the group consisting of cannabigerolic acid (CBGA), cannabidivarin (CBDV), cannabichromenic acid (CBCA), cannabidivarinic acid (CBDVA), and tetrahydrocannabivarin (THCV).

29. The cannabinoid composition according to claim 28, comprising CBDV and at least one additional cannabinoid selected from the group consisting of CBGA, CBCA, CBDVA, and THCV.

30. The cannabinoid composition according to claim 28, consisting essentially of at least two cannabinoids selected from the group consisting of CBDV, CBGA, CBCA, CBDVA, and THCV.

31. The cannabinoid composition according to claim 29, comprising at least CBDV and CBGA.

32. The cannabinoid composition according to claim 31, consisting essentially of CBDV and CBGA.

33. The cannabinoid composition according to claim 28, wherein the % w/w ratio of the at least two cannabinoids in the composition is between 1:10 and 10:1.

34. The cannabinoid composition according to claim 33, wherein the at least two cannabinoids are CBDV and CBGA, and wherein the % w/w ratio of CBDV and CBGA in the composition is between 1:10 and 10:1.

35. The cannabinoid composition according to claim 34, wherein the % w/w ratio of CBDV and CBGA in the composition is 1:1.

36. The cannabinoid composition according to claim 28, wherein the cannabinoid is selected from the group consisting of a synthetic cannabinoid, an isolated cannabinoid derived from a cannabis plant, a cannabis plant extract comprising at least 50% (w/w) of the cannabinoid and a combination thereof.

37. The cannabinoid composition according to claim 28, wherein the cannabinoids in the composition exhibit a synergistic anti-cancer effect.

38. The cannabinoid composition according to claim 28, further comprising a chemotherapeutic agent.

39. The cannabinoid composition according to claim 28, wherein the composition is substantially free of a cannabinoid other than CBGA, CBDV, CBCA, CBDVA, and/or THCV.

40. The cannabinoid composition according to claim 28, wherein the cannabinoids in the composition are effective in inhibiting cancer cells' proliferation, inducing cancer cells' death, or a combination thereof.

41. A method for treating cancer comprising administering to a subject in need a cannabinoid composition as defined in claim 28.

42. The method according to claim 41, wherein the cancer is a solid cancer.

43. The method according to claim 42, wherein the solid cancer is selected from colorectal cancer, breast cancer, lung cancer, and skin cancer.

44. The method according to claim 41, further comprises administering to the subject a chemotherapeutic agent.

45. The method according to claim 44, wherein the chemotherapeutic agent is an antimetabolite drug, a DNA alkylating agent, a platinum compound, a Topoisomerase inhibitor, a Tyrosine kinase inhibitor, a vincalkaloid, a taxane, an antibiotic, and a combination thereof.

46. The method according to claim 44, wherein the chemotherapeutic agent is selected from the group consisting of Capecitabine, Docetaxel, Epirubicin, 5-Fluorouracil, Oxaliplatin, Irinotecan and a combination thereof.

47. The method according to claim 41, which is prescribed for oral administration.
